(19) United States
(12) Patent Application Publication
(10) Pub. No.: US 2024/0207101 A1
(43) Pub. Date: Jun. 27, 2024

(54) METHOD AND APPARATUS FOR BIOPRINTING

(71) Applicant: The Catholic University of America, Washington, DC (US)

(72) Inventors: Christopher Brian RAUB, Washington, DC (US); George NEHMETALLAH, Washington, DC (US); Santiago O. CORREA, Washington, DC (US)

(21) Appl. No.: 18/544,787

(22) Filed: Dec. 19, 2023

Related U.S. Application Data
(60) Provisional application No. 63/434,538, filed on Dec. 22, 2022.

(51) Int. Cl. A61F 13/02; A61L 15/18; A61L 15/32; A61L 15/60; C12M 1/12; C12M 1/34; C12M 3/00; C12N 5/077

(52) U.S. Cl. CPC A61F 13/0289; A61L 15/18; A61L 15/325; A61L 15/60; C12M 21/08; C12M 25/14; C12M 41/36; C12N 5/0656; C12N 2513/00; C12N 2533/30; C12N 2533/54; C12N 2537/10

(57) ABSTRACT

A method and apparatus including an inverted microscope sample stage, a photomask, a light diffuser, a light pipe, and a light source. The light source is confocal with an inverted microscope, and the apparatus is configured to produce a ruthenium-mediated photocrosslink pattern into a subject. The photomask may be disposed between the inverted microscope sample stage and the light diffuser. The light diffuser may be disposed between the photomask and the light pipe. The light pipe may be disposed between the light diffuser and the light source. The light source may be disposed above the light pipe,

### CROSS-REFERENCE TO RELATED APPLICATIONS

This application claims benefit of priority to U.S. Provisional Patent Application No. 63/434,538, entitled “METHOD AND APPARATUS FOR BIOPRINTING BY PHOTO-CROSSLINKING COLLAGEN AND GELATIN USING LIQUID CRYSTAL DISPLAYS, VISIBLE LIGHT AND CONTROL OF HYDROGEL SWELLING” filed Dec. 22, 2022. The entire contents and disclosures of these patent applications are incorporated herein by reference in their entirety.

### BACKGROUND

### Field of the Invention

The present disclosure relates generally to a method and apparatus for bioprinting by photo-crosslinking bioinks, including collagen, gelatin, polyethylene diacrylate, and methacrylated biopolymers. In some cases employed techniques utilize liquid crystal displays, visible light, and control of hydrogel swelling.

### Background of the Invention

Biofabrication is the marriage of biology and microfabrication (1), in which aspects of fabrication and design from semiconductor chip technologies are applied to biomaterials and in vitro living systems. Researchers have adapted biofabrication approaches for use in tissue repair and regeneration (2), pharmaceutical testing and disease modeling on a chip (3), in devices (4) and interfacing biotic and abiotic systems (5). Biofabrication also borrows from techniques such as microcontact printing (6), photolithography (7), and tissue engineering with engineered peptides and proteins conjugated to hydrogel scaffolds (8). For biofabricated tissue constructs, the design typically occurs first, followed by cell culture and construct development, maturation, and remodeling through the activity of cells, enzymes, and mechanical forces (9, 10). Two major issues with engineered tissues that could be solved through biofabrication approaches are failure to recapitulate vital functions of native tissues (11), and excessive tissue remodeling with invasion of cells beyond their desired boundaries (12). There is a need for biofabrication techniques that offer the possibility of greater control over cell, tissue, and organ function by engineering microstructural complexity across multiple spatial scales, more closely recapitulating native tissue microarchitecture, mechanics and biochemistry (13).

### SUMMARY

According to a first broad aspect, the present disclosure provides an apparatus comprising: an inverted microscope sample stage; a photomask; a light diffuser; a light pipe; and a light source, wherein the light source is confocal with the inverted microscope, and wherein the apparatus is configured to produce a ruthenium-mediated photocrosslink pattern into a subject.

According to a second broad aspect, the present disclosure provides a method of bioprinting comprising: bioprinting a photocrosslink pattern to a subject using an apparatus comprising: an inverted microscope sample stage; a photomask; a light diffuser; a light pipe; and a light source, wherein the light source is confocal with an inverted microscope, and wherein the apparatus produces a ruthenium-mediated photocrosslink pattern into the subject.

According to a third broad aspect, the present disclosure provides a method of wound treatment comprising: bioprinting a photocrosslink pattern to a subject using an apparatus comprising: an inverted microscope sample stage; a photomask; a light diffuser; a light pipe; and a light source, wherein the light source is confocal with an inverted microscope, wherein the apparatus produces a ruthenium-mediated photocrosslink pattern into the subject, and applying the photocrosslink patterned subject to an individual.

According to a fourth broad aspect, the present disclosure provides a kit comprising: an effective amount of a material, wherein the material comprises: a photocrosslink patterned subject produced by an apparatus comprising: an inverted microscope sample stage; a photomask; a light diffuser; a light pipe; and a light source, wherein the light source is confocal with an inverted microscope, and wherein the apparatus is configured to produce a ruthenium-mediated photocrosslink pattern into the subject.

According to a fifth broad aspect, the present disclosure provides a wound dressing or wound healing agent comprising: a photocrosslink patterned subject produced by an apparatus comprising: an inverted microscope sample stage; a photomask; a light diffuser; a light pipe; and a light source, wherein the light source is confocal with an inverted microscope, and wherein the apparatus is configured to produce a ruthenium-mediated photocrosslink pattern into the subject.

According to a sixth broad aspect, the present disclosure provides a pharmaceutical composition comprising: a photocrosslink patterned subject produced by an apparatus comprising: an inverted microscope sample stage; a photomask; a light diffuser; a light pipe; and a light source, wherein the light source is confocal with an inverted microscope, and wherein the apparatus is configured to produce a ruthenium-mediated photocrosslink pattern into the subject.

According to a seventh broad aspect, the present disclosure provides a device for delivering a photocrosslink patterned subject to an individual, wherein the photocrosslink patterned subject is produced by an apparatus comprising: an inverted microscope sample stage, a photomask, a light diffuser, a light pipe, and a light source, wherein the light source is confocal with an inverted microscope, and wherein the apparatus is configured to produce a ruthenium-mediated photocrosslink pattern into the subject.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The accompanying drawings, which are incorporated herein and constitute part of this specification, illustrate exemplary embodiments of the invention, and, together with the general description given above and the detailed description given below, serve to explain the features of the invention.

FIGS. 1A-1B illustrate schematic and photographs of the photocrosslink patterning device, including (i) the light emitting diode array, (ii) the graphic LCD screen, and (iii) blue light pattern imaged by the microscope, according to one embodiment of the present disclosure.

FIGS. 2A-2C illustrate crosslinked gelatin patterns designed with an LCD array as photomask, of a dot array (FIG. 2A), an academic logo and text (FIG. 2B), and a maze pattern (FIG. 2C), according to one embodiment of the present disclosure. Patterns are projected through (i) 20×, (ii) 40×, and (iii) 60× focusing objectives.

FIGS. 3A-3C illustrate pattern profiles across the crosslinked dot pattern from top (T) to bottom (B), indicated by the red rectangular region of interest, formed with 20× (FIG. 3A), 40× (FIG. 3B), and 60× (FIG. 3C) focusing objectives and profiles are plotted to the right of each image, according to one embodiment of the present disclosure.

FIGS. 4A-4D illustrate collagen crosslinked using the LCD photomask for 60 s (FIG. 4A), 120 s (FIG. 4B), 180 s (FIG. 4C), and 240 s (FIG. 4D), according to one embodiment of the present disclosure. Some pattern contrast features are visible in brightfield (FIGS. 4A.i-4D.i) and phase contrast (FIGS. 4A.ii-4D.ii) images.

FIGS. 5A-5F illustrate patterned photoconjugation of GFP to gelatin (FIGS. 5A, 5C.i-5F.i) and collagen (FIGS. 5B, 5C.ii-5F.ii and 5C.iii-5F.iii) hydrogels at varying crosslinking exposure times, according to one embodiment of the present disclosure. GFP patterns in gelatin are emplaced with an LCD photomask; for collagen, the LCD apparatus is removed, and patterning is achieved using the focused LED array image.

FIGS. 6A-6C illustrate time-lapse monitoring of collagen hydrogels crosslinked with 20× (FIGS. 6A and 6C) and 60× (FIG. 6B) focusing objectives and subsequently rinsed (FIGS. 6A-6B), or unrinsed before incubation with nutrient media containing 10% fetal bovine serum (FIG. 6C), according to one embodiment of the present disclosure. Phase contrast micrographs are collected serially, with timepoints of 1, 8, 20, and 48 hours indicated (I-iv, respectively).

FIGS. 7A-7B illustrate time-lapse microscopy of fibroblasts cultured on LED array patterns emplaced into collagen hydrogels with 20× (FIG. 7A) and 60× (FIG. 7B) focusing objectives, according to one embodiment of the present disclosure. Timepoints of 0, 1, 8, and 16 hours (i-iv, respectively) are indicated.

FIGS. 8A-8B illustrate fibroblasts cultured on top of collagen hydrogels patterned with an LED array image through a 20× (FIG. 8A) and 60× focusing objective (FIG. 8B) and imaged using (i) phase contrast and (ii) epifluorescence microscopy, the latter after fixation, permeabilization, and staining of actin and DNA, according to one embodiment of the present disclosure.

FIGS. 9A-9B illustrate micrographs of fibroblasts seeded onto a collagen hydrogel patterned with matrix defects created by hydrogel swelling after photopatterning. Repeated phase contrast microscopy imaging after 1 (FIG. 9A.i), 2 (FIG. 9A.ii), 3 (FIG. 9A.iii), and 4 days (FIG. 9A.iv) of cell culture, according to one embodiment of the present disclosure. A confocal micrograph of fibroblasts stained with fluorescent probes against actin (green) and DNA (red), cultured on the patterned hydrogel (FIG. 9B).

FIGS. 10A-10B illustrate a modular platform for dynamic focal screening, consisting of blue light LED array, thin-film-transistor liquid crystal display screen, a control board, a camera adaptable to the microscope, and camera control/image processing/TFT-LCD/LED control software, according to one embodiment of the present disclosure.

FIG. 10C illustrates a fluorescence signal from collagen after conjugation of green fluorescent protein (GFP) to the collagen network using a Ruthenium crosslinker (XL), blue light exposure, and extensive rinsing (hv+rinse), according to one embodiment of the present disclosure.

FIG. 10D illustrates simulated data for a desired map of the endogenous optical signal from native tissue or some other idealized, imposed optical map, according to one embodiment of the present disclosure.

FIG. 11 illustrates a concept of directed microstructural engineering of tissue constructs, according to one embodiment of the present disclosure.

FIG. 12 illustrates conditional generative adversarial neural nets (cGAN) to relate the light configuration to the reference (ground truth) birefringence and phase images, according to one embodiment of the present disclosure.

FIG. 13 illustrates an SLM/DMD based bi-telecentric holographic microscope, according to one embodiment of the present disclosure.

FIG. 14 illustrates an injectable device for delivering a photocrosslink patterned subject, according to one embodiment of the present disclosure.

FIG. 15 illustrates a patch device for delivering a photocrosslink patterned subject, according to one embodiment of the present disclosure.

FIG. 16 illustrates a graph of change in hydrogel wet weight versus photocrosslinking exposure time, according to one embodiment of the present disclosure. Greater photocrosslinking reduces the change in wet weight of hydrogels.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present disclosure, the term “amino acid” refers to the molecules composed of terminal amine and carboxylic acid functional groups with a carbon atom between the terminal amine and carboxylic acid functional groups sometimes containing a side chain functional group attached to the carbon atom (e.g., a methoxy functional group, which forms the amino acid serine). Typically, amino acids are classified as natural and non-natural. Examples of natural amino acids include glycine, alanine, valine, leucine, isoleucine, proline, tyrosine, tryptophan, serine, threonine, cysteine, methionine, asparagine, glutamine, lysine, arginine, histidine, aspartate, and glutamate, among others. Examples of non-natural amino acids include L-3,4-dihydroxyphenylalanine, 2-aminobutyric acid, dehydroalanine, g-carboxyglutamic acid, carnitine, gamma-aminobutyric acid, hydroxyproline, and selenomethionine, among others. Examples for non-natural amino acids that are routinely incorporated at JPT are: D-amino acids, homo amino acids, N-methyl amino acids, alpha-methyl amino acids, beta (homo) amino acids, gamma amino acids, helix/turn stabilizing motifs, backbone modifications (e.g. peptoids). JPT is also able to incorporate a wide range of unusual amino acids found in nature. Prominent examples are: hydroxyproline (Hyp), beta-alanine, citrulline (Cit), ornithine (Orn), norleucine (Nle), 3-nitrotyrosine, nitroarginine, pyroglutamic acid (Pyr). In the context of this specification, it should be appreciated that the amino acids may be the L-optical isomer or the D-optical isomer.

For purposes of the present disclosure, a value or property is “based” on a particular value, property, the satisfaction of a condition, or other factor, if that value is derived by performing a mathematical calculation or logical decision using that value, property, or other factor.

For purposes of the present disclosure, the term “3D printer” refers to a device which makes a physical object from a three-dimensional digital model which may typically include laying down many thin layers of a material in succession. In some embodiments, 3D printer, or additive manufacturing is the construction of a three-dimensional object such as from a CAD model or a digital 3D model that is converted into a G-code that provides the pathway to define the printed structure. It can be done in a variety of processes in which material is deposited, joined, or solidified under computer control, with the material being superposed layer-by-layer and added together (such as viscous-liquids or compressed-powder grains being fused), typically layer by layer.

For purposes of the present disclosure, the term “bioink” refers to any synthetic or natural polymer used to form structures and patterns in bioprinting. In some embodiments, bioinks may include collagen, gelatin, polyethylene diacrylate, and methacrylated biopolymers.

For purposes of the present disclosure, the term “biofabrication” refers to a branch of biotechnology specializing in the research and development of biologically engineered processes for the automated production of biologically functional products through bioprinting or bio assembly and subsequent tissue maturation processes; as well as techniques such as directed assembly, which employs localized external stimuli guide the fabrication process; enzymatic assembly, which utilizes selective biocatalysts to build macromolecular structures; and self-assembly, in which the biological material guides its own assembly according to its internal information.

For purposes of the present disclosure, the term “biomacromolecules” refer to large biological molecules typically polymers, such as nucleic acids, proteins, and carbohydrates, that are made up of monomers linked together. They may have sizes of at least 800 Daltons, high molecular weights, and typically complex structures.

For purposes of the present disclosure, the term “biomolecule” refers to the conventional meaning of the term biomolecule, i.e., a molecule produced by or found in living cells, e.g., a protein, a carbohydrate, a lipid, a phospholipid, a nucleic acid, etc.

For purposes of the present disclosure, the term “bioprinting” refers to a technology where bioinks and biomaterials, mixed with cells, are printed, often to construct living tissue models. By way of example only, bioprinting may produce living tissue, bone, blood vessels and, potentially, whole organs for use in medical procedures, training, and testing.

For purposes of the present disclosure, the term “collagen” refers to the main structural protein in the extracellular matrix found in the body's various connective tissues.

For purposes of the present disclosure, the term “comprising”, the term “having”, the term “including.” and variations of these words are intended to be open-ended and mean that there may be additional elements other than the listed elements.

For purposes of the present disclosure, the term “computer” refers to any type of computer or other device that implements software including an individual computer such as a personal computer, laptop computer, tablet computer, mainframe computer, minicomputer, etc. A computer also refers to electronic devices such as an electronic scientific instrument such as a spectrometer, a smartphone, an eBook reader, a cell phone, a television, a handheld electronic game console, a videogame console, a compressed audio, or video player such as an MP3 player, a Blu-ray player, a DVD player, etc. In addition, the term “computer” refers to any type of network of computers, such as a network of computers in a business, a computer bank, the Cloud, the Internet, etc. Various processes of the present disclosure may be carried out using a computer. Various functions of the present disclosure may be performed by one or more computers.

For purposes of the present disclosure, the term “confocal” refers to having the same focus.

For purposes of the present disclosure, the term “conjugation” refers to linking together.

For purposes of the present disclosure, the term “crosslink” refers to a bond or a short sequence of bonds that links one polymer chain to another.

For purposes of the present disclosure, the term “3-D bioprinting” refers to the utilization of 3D printing-like techniques to combine cells, growth factors, bio-inks, and/or biomaterials to fabricate biomedical parts that imitate natural tissue characteristics, form functional biofilms, and assist in the removal of pollutants.

For purposes of the present disclosure, the term “data” means the reinterpretable representation of information in a formalized manner suitable for communication, interpretation, or processing. Although one type of common type data is a computer file, data may also be streaming data, a web service, etc. The term “data” is used to refer to one or more pieces of data.

For purposes of the present disclosure, the term “deep learning” refers to the subset of machine learning methods based on artificial neural networks with representation learning.

For purposes of the present disclosure, the term “endogenous tissue optical signals” refers to optical signals generated by one or more tissue components, including cells, extracellular matrix components, and extracellular soluble and insoluble factors, without the addition of exogenous optical labels or probes. Examples of endogenous optical signals include autofluorescence, birefringence, optical phase, optical absorption/attenuation, optical diattenuation, second harmonic generation signal, two-photon autofluorescence, darkfield optical scattering signal, circular dichroism, optical Kerr effect, and photoacoustic signals.

For purposes of the present disclosure, the term “digital micro-mirror devices” refers to a matrix of micro mirrors that by changing orientation allows the light to be diverted in a controlled way to generate a pattern.

For purposes of the present disclosure, the term “epifluorescence microscopy” refers to a microscope which has a feature of a parallel beam of light that is passed directly upwards through the sample, maximizing the amount of illumination.

For purposes of the present disclosure, the term “effective amount” or “effective dose” or grammatical variations thereof refers to an amount of an agent sufficient to produce one or more desired effects. The effective amount may be determined by a person skilled in the art using the guidance provided herein.

For purposes of the present disclosure, the term “elastic modulus” refers to the unit of measurement of an object's or substance's resistance to being deformed elastically (i.e., non-permanently) when a stress is applied to it.

For purposes of the present disclosure, the term “gel” and “hydrogel” are used interchangeably. These terms refer to a biphasic material such as a network of polymer chains, entrapping water, or other aqueous solutions, such as physiological buffers, of 10% or more by weight.

For purpose of the present disclosure, the term “gelatin” refers to a collection of peptides and proteins produced by partial hydrolysis of collagen extracted from the skin, bones, and connective tissues of animals such as domesticated cattle, chicken, pigs, and fish.

For purpose of the present disclosure, the term “green fluorescent protein” refers to a protein that exhibits bright green fluorescence when exposed to light in the blue to ultraviolet range.

For purposes of the present disclosure, the term “halogen” refers to a light source which has a halogen, such as iodine or bromine.

For purposes of the present disclosure, the term “hydrogels” refers to a biphasic material, a mixture of porous, permeable solids and at least 10% by weight or volume of interstitial fluid composed completely or mainly by water.

For purposes of the present disclosure, the term “hydrogel scaffold” or “tissue scaffold” refers to a structure to provide bulk and mechanical structure to a tissue construct.

For purposes of the present disclosure, the term “individual” refers to an entity which is the object of treatment, observation, or experiment. By way of example only, a “individual” may be, but is not limited to: a human, a mammal, a reptile, a bird, a fish, an amphibian, and an invertebrate.

For purposes of the present disclosure, the term “inverted microscope” refers to a microscope with the objective lens placed below the stage on which the sample is located, allowing the sample to be observed from below.

For purposes of the present disclosure, the term “in vitro” refers to the outside of the living body and in an artificial environment.

For purposes of the present disclosure, the term “laser” is a device that emits light through a process of optical amplification based on the stimulated emission of electromagnetic radiation.

For purposes of the present disclosure, the term “LED” refers to a light-emitting diode which is a semiconductor device that emits light when current flows through it.

For purposes of the present disclosure, the term “light” unless specified otherwise, refers to any type of electromagnetic radiation. Although, in the embodiments described below, the light that is incident on the gratings or sensors is visible light, the light that is incident on the gratings or sensors of the present disclosure may be any type of electromagnetic radiation, including infrared light, ultraviolet light, etc., that may be scattered by a grating or sensor. Although, in the embodiments described below; the light that is scattered from the gratings or sensors and detected by a detector is visible light, the light that is scattered by a grating or sensor of the present disclosure and detected by a detector of the present disclosure may be any type of electromagnetic radiation, including infrared light, ultraviolet light, etc. that may be scattered by a grating or sensor.

For purposes of the present disclosure, the term “light diffuser” refers to any material that diffuses or scatters light in some manner to transmit soft light.

For purposes of the present disclosure, the term “light pipe” refers to an optical fiber or a solid transparent plastic rod for transmitting light lengthwise.

For purposes of the present disclosure, the term “light source” refers to a source of incident light that is scattered by a grating or sensor of the present disclosure. In one embodiment of the present disclosure, a light source may be part of an apparatus of the present disclosure. In one embodiment a light source may be light present in the environment of a sensor or grating of the present disclosure. A light source may even be the ambient light of a room in which a grating or sensor of the present disclosure is located. Examples of a light source include a laser, a light-emitting diode (LED), an incandescent light bulb, a compact fluorescent light bulb, a fluorescent light bulb, etc.

For purposes of the present disclosure, the term “mercury-arc lamp” refers to a gas-discharge lamp that uses an electric arc through vaporized mercury to produce light.

For purposes of the present disclosure, the term “metallurgical microscope” refers to a microscope for looking at cross-sections of metal targets (metallurgical mounts). Typically inverted, these metallurgical microscopes employ high-resolution objective lenses with very short working distances.

For purposes of the present disclosure, the term “microstructure” refers to a structure having at least one dimension smaller than 1 mm. A nanostructure is one type of microstructure.

For purposes of the present disclosure, the term “microscopy” refers to the technical field of using microscopes to view objects and areas of objects that cannot be seen with the naked eye.

For purposes of the present disclosure, the term “microscope sample stage” refers to a stage where an object is placed for observation.

For purposes of the present disclosure, the term “monochrome LCD” refers to a type of liquid crystal display (LCD) that may only display images in a single color.

For purposes of the present disclosure, the term “nanostructure” refers to a structure having at least one dimension on the nanoscale, i.e., a dimension between 0.1 and 100 nm.

For purposes of the present disclosure, the term “optical contrast” is a normalized difference between the intensities of light reflected by sample and surrounding substrate.

For the purposes of the present invention, the term “patch” refers to an adhesive patch that is placed on the skin or surfaces.

For purposes of the present disclosure, the term “pattern” refers to a design.

For purposes of the present disclosure, the term “photocrosslink” refers to a photoinduced formation of a covalent bond between two macromolecules or between two different parts of one macromolecule.

For purposes of the present disclosure, the term “photoinitiator” refers to a molecule that creates reactive species such as free radicals, cations or anions when exposed to radiation such as UV or visible sources such as blue light, for example.

For purposes of the present disclosure, the term “photomask” refers to an opaque plate with transparent areas that allow light to shine through in a defined pattern.

For purposes of the present disclosure, the term “photopatterning” refers to the production of a photochemical etching on the surface of a semiconductor.

For purposes of the present disclosure, the term “proteins” refer to large biomolecules and macromolecules that comprise one or more long chains of amino acid residues.

For purposes of the present disclosure, the term “room temperature” refers to a temperature of from about 20° C. to about 25° C.

For purposes of the present disclosure, the term “ruthenium-mediated” means ruthenium is being utilized in the scope of the invention.

For purposes of the present disclosure, the term “spatial light modulation” refers to an optical device that imposes some form of spatially varying modulation on a beam of light.

For purposes of the present disclosure, the term “time” refers to a component of a measuring system used to sequence events, to compare the durations of events and the intervals between them, and to quantify the motions of objects. Time may be considered one of the few fundamental quantities and is used to define quantities such as velocity. An operational definition of time, wherein one says that observing a certain number of repetitions of one or another standard cyclical event (such as the passage of a free-swinging pendulum) constitutes one standard unit such as the second, has a high utility value in the conduct of both advanced experiments and everyday affairs of life. Temporal measurement has occupied scientists and technologists and was a prime motivation in navigation and astronomy. Periodic events and periodic motion have long served as standards for units of time. Examples include the apparent motion of the sun across the sky, the phases of the moon, the swing of a pendulum, and the beat of a heart. Currently, the international unit of time, the second, may be defined in terms of radiation emitted by cesium atoms.

For purposes of the present disclosure, the term “tissue engineering” refers to a biomedical engineering discipline that uses a combination of cells, engineering, materials methods, and suitable biochemical and physicochemical factors to restore, maintain, improve, or replace different types of biological tissues.

For purposes of the present disclosure, directional terms such as “top,” “bottom,” “upper,” “lower,” “above.” “below;” “left,” “right,” “horizontal,” “vertical,” “up,” “down,” etc., are used merely for convenience in describing the various embodiments of the present disclosure. The embodiments of the present disclosure may be oriented in various ways. For example, the diagrams, apparatuses, etc., shown in the drawing figures may be flipped over, rotated by 90° in any direction, reversed, etc.

### Description

While the invention is susceptible to various modifications and alternative forms, specific embodiment thereof has been shown by way of example in the drawings and will be described in detail below. It should be understood, however that it is not intended to limit the invention to the particular forms disclosed, but on the contrary, the invention is to cover all modifications, equivalents, and alternatives falling within the spirit and the scope of the invention.

A purpose of the disclosed invention is to allow greater control of microstructure in cultured tissue constructs, for in vitro assays and implantation. This is a new process with associated products (modular platform attached to microscopes and reagent kit).

3-D bioprinting is a family of methods that write microscale patterns of cells, bioactive molecules, and biomaterials to generate tissue-like constructs for biomedical applications. Biomaterials materials may include natural biomacromolecules, such as alginate, gelatin, collagen, and chitosan, and synthetic biomaterials such as polylactic acid, poly-8-caprolactone, and others. Tissue 3-D morphology and structure are derived from medical imaging data (computed tomography scans, or magnetic resonance imaging, for example) translated into computer-aided drawing files. An actuator (stepper motors, for example) scans an extrusion nozzle across a surface, and the construct components are extruded in a layer-by-layer fashion. After the initial imposed scaffold geometry and cell organization, tissue development occurs by autonomous self-assembly, directed by embedded cells' genetic programming.

Alternatively to 3-D bioprinting with stepper motors, various photolithography techniques exist for imposing photopolymerized structures in biocompatible hydrogels. These typically use UV or another light source and a photomask, or else direct scanned lasers or other structured light into photopolymerizable systems.

One problem with 3-D bioprinting is that very little direct control over tissue remodeling exists after the initial imposed geometry. While there are end goals for tissue function, it is difficult to know when the tissue constructs have achieved a final state that allows performance of those functions.

A second problem with 3-D bioprinting is lack of sub-micron resolution in printed microstructures. The “bioink” containing cells must transition from liquid to solid rapidly, while the extrusion process also limits the resolution of features that can be printed.

Further, photopolymerization techniques often require either ultraviolet light, which is toxic to cells, or else do not work with native biopolymer systems, such as acid-soluble collagen hydrogels.

Perfusion of 3-D bioprinted constructs with blood or a blood substitute is technically challenging, often resulting in tissue mechanical failure, uneven perfusion and tissue necrosis, making the constructs unacceptable for use as implantable tissues.

The disclosed method of directed microstructural engineering solves the problem of poor control over tissue remodeling in vitro by guiding the tissue toward a desired outcome based on images of native tissue, using feedback control. Screened patterns of light direct photochemistry to bind or release bioactive molecules in areas where the tissue construct's microstructure is far from native tissue. Transfer functions for microstructural alterations resulting from the photochemical reactions determine the extent of local tissue remodeling, toward a desired microstructure, by a mechanism in which local cells and local extracellular matrix proteins only are affected and respond in a predictable manner. This will allow tissue construct microstructure to very nearly exactly resemble native tissue microstructure to the extent that endogenous optical signals (such as birefringence, optical phase, and second harmonic generation signal) converge.

Such an approach can lead to the development of well-perfused mechanically stable tissue constructs by creating collagen microstructurally-reinforced channels in a hydrogel, using photochemistry to guide the microstructure and also the mechanics of the tissue construct until desired levels have been achieved. The photocrosslinking-derived conjugation and release of proteases, growth factors, chemokines, bioactive peptides, nucleic acids, and extracellular matrix molecules to each other allows mimicry of tissue remodeling during development, growth and maturation, leading to improved tissue mechanics and less frequent mechanical failure.

Endogenous optical signals derive from molecular organization at the single molecule through the nanoscale and up to the macroscale. Thus this method provides control over biological molecules with greater precision than current techniques, as matching endogenous optical signals (birefringence, phase, and others) implies a convergence of molecular scale organization.

The main disadvantage of this process is that it is likely to be slow, depending on cell activity to remodel tissue constructs with directed photochemistry to guide and perhaps accelerate the remodeling process. In vivo, many tissues take weeks to months to develop, while maturation is a process that can take years. Here, directed photochemistry may provide a way to overcome this limitation, by accelerating extracellular reactions that promote tissue remodeling and maturation. Directed light patterns could also locally turn on selected genes to provide local control over gene expression through optogenetics, which would also accelerate tissue development.

A second disadvantage is that endogenous optical signals recorded by most microscopes lose resolution and contrast in thick tissues. To circumvent this, directed microstructural engineering will be pursued in two-photon laser scanning microscopy platforms. Two-photon laser scanning microscopy builds 3-D images with high depth as well as lateral resolution by restricting signal generation to the focal volume of a scanned laser. The second harmonic generation signal at 380-450 nm wavelengths provides ideal energies for photochemistry with Ruthenium based crosslinkers and would direct the photochemistry with high, sub-micron resolution.

The best model for practicing directed microstructural engineering is to build cell-seeded, collagen hydrogel constructs layer-by-layer on an incubated polarized light or digital holographic microscope, or other microscope, using: 1) A modular platform for dynamic focal screening, consisting of blue light LED array, thin-film-transistor liquid crystal display screen, a control board, a camera adaptable to the microscope, and camera control/image processing/TFT-LCD/LED control software; 2) A reagent kit consisting of ruthenium-based photocrosslinkers and photorelease reagents, and bioactive molecules that interact with cells and extracellular matrix; and 3) A desired map of endogenous optical signal from native tissue or some other idealized, imposed optical map.

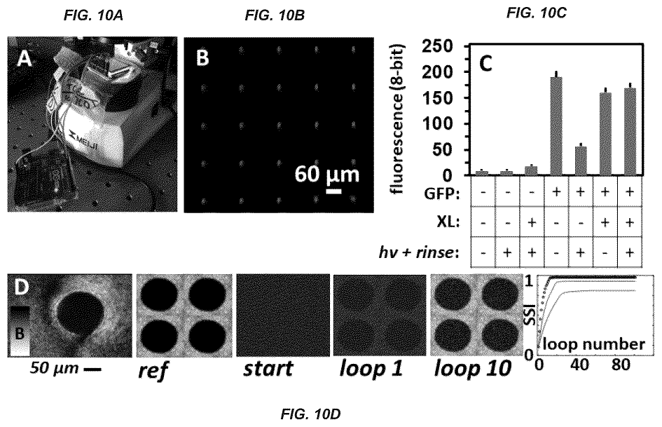

FIGS. 10A-10D illustrate data in support of feasibility and operability of the invention. Data for 1) are provided, in FIGS. 10A-10B. Data for 2) are provided, in FIG. 10C. Green fluorescent protein (GFP) is crosslinked to collagen by Ruthenium (XL), activated by blue light (hv), and stable to rinsing with saline (rinse). Simulated data for 3) are provided, in FIG. 10D. A real birefringence map of a collagen reinforced pore is made into a repeating map to generate a pore array (ref). Low signal from an initially random field (start) develops toward ref using closed-loop feedback. The structural similarity index (SSI) becomes higher with an increasing number of feedback loops.

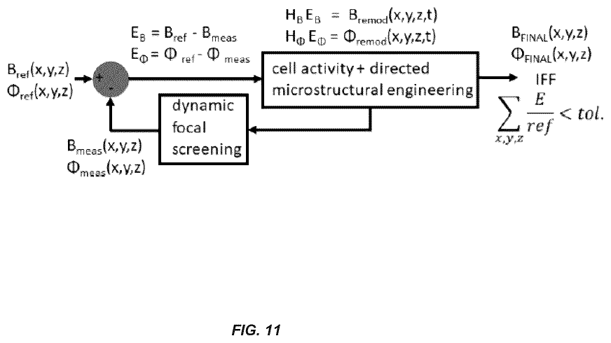

It is anticipated that general rules for directed microstructural engineering exist, such that most cell types embedded in collagen hydrogels respond similarly to pattered conjugation and/or release of bioactive molecules to/from the collagen fibril network. Such rules may be inferred from cell-seeded collagen gel responses to exogenously added bioactive agents in the absence of patterning. However, the rules for directed microstructural engineering are applied in this invention in a novel fashion, to direct endogenous optical signals toward a desired pattern using closed-loop feedback (FIG. 11).

FIG. 11 illustrates a concept of directed microstructural engineering of tissue constructs, according to one embodiment of the present disclosure. Microstructure is guided to develop during culture by measuring endogenous optical signals (birefringence, Bₘₑₐₛ, and phase, Φₘₑₐₛ), comparing to desired signal maps B_{ref} and Φ_{ref}, and applying transfer functions |H_{B}, H_{Φ}|<1 until measured signals converge to reference by some tolerance, tol. Microscopy-based dynamic focal screens provide spatiotemporal control (FIG. 11).

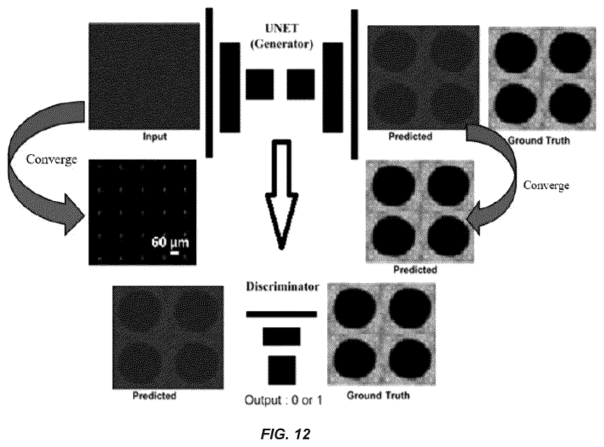

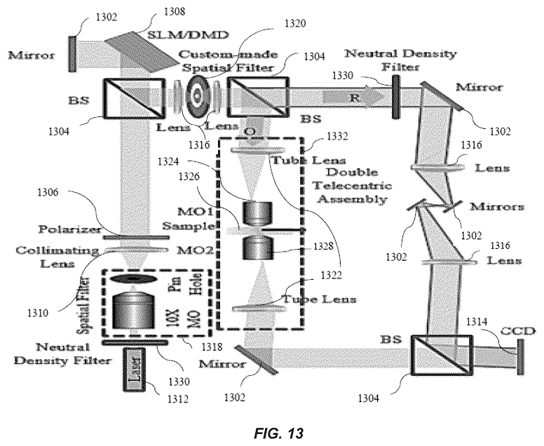

Alternatives to the previously described embodiments may include: 1) any other way of directing light into polymeric scaffolds, including the use of spatial light modulators and digital micro-mirror devices, any light source that induces a chemical reaction in the scaffold and/or in the cells embedded in the scaffold that could lead to altered microstructure, and any way to receive optical signals from the scaffold, useful for feedback; 2) any kind of photosensitive compound that interacts with cells and microstructural elements in a construct to induce a change in the biological features of the living system; 3) any sort of micrograph, image, or optical signal from the construct system, and 4) any kind of feedback controller, including the use of deep learning conditional adversarial networks and other kinds of deep learning algorithms. In exemplary designs, some of the alternative approaches are illustrated in FIGS. 12-13.

FIG. 12 illustrates conditional generative adversarial neural nets (cGAN) to relate the light configuration to the reference (ground truth) birefringence and phase images, according to one embodiment of the present disclosure. Disclosed embodiments may first start with a random input and then the network will learn how to converge to the right configuration for a certain result.

FIG. 13 illustrates an SLM/DMD based bi-telecentric holographic microscope, in one exemplary configuration of the disclosed invention. The DMD or SLM can rotate the beam and can imprint patterns of light configurations on the sample.

Embodiments of the disclosed microscope (FIG. 13) may include mirror (1302), BS (1304), polarizer (1306), collimating lens (1310), laser (1312), neutral density filter (1330), spatial filter (1318), SLM/DMD (1308), lens (1316), custom-made spatial filter (1320), MO1 (1324), sample (1326), MO2 (1328), tube lens (1322), CCD (1314), and double telecentric assembly (1332).

The light from the laser (1312) is first attenuated with a variable neutral density filter (1330), then the beam is cleaned and made circular Gaussian shape by a spatial filter (1318). The spatial filter (1318) is situated at the front focal length of a collimating lens (1310). After the collimating lens (1310) the beam is collimated and is transmitted through a linear polarizer (1306). Then the beam travels through a first beam splitter (1304) before hitting the Spatial light modulator/DMD device (1308). The DMD beam then hits a mirror (1302). The job of the SLM/DMD is to imprint a pattern on the beam and or to bend/steer the beam into different directions. Then the beam is also focused into another custom-made spatial filter (1320) to eliminate higher order diffraction patterns that result from the SLM/DMD device (1308). Then the beam hits a second beam splitter (1304). One part of the beam called the object beam travels through a double telecentric assembly (1332). In this assembly there is a first tube lens (1322) then a first microscope objective MO1 (1324) and the biological sample (1326), then another MO2 (1328) and another tube lens 2 (1322). At the output of the double telecentric system the object beam hits a mirror (1302) and a third beam splitter (1304). The second part of the beam that got transmitted from the second beam splitter (1304) is called the reference beam. This beam also attenuated through another neutral density filter (1330) and gets reflected by a mirror (1302). This beam also is focused by a lens (1316) to a first mirror (1302) and then it diffracts to a second mirror (1302) and then it is collimated back again by a lens (1316). The two mirror/lens system in the reference beam path is used to make sure that the path length of the object beam and the reference beam are the same. Then the reference beam is collimated by lens (1316) and hits the third beam splitter (1304) where it is reflected and gets combined with the transmitted object beam. Both beams hit the CCD imaging system (1314).

The disclosed configuration may provide new features or improvements over known methods including: 1) the use of optical signals to direct local biological activity in tissue constructs during culture. 2) Photocrosslinking and photorelease guided by a dynamic focal screen of light—allowing flexible, rapid photopatterning of molecules and molecular activity in thin tissue constructs. 3) Adaptability to sub-micron, 3-D resolved photopatterning using second harmonic generation (SHG) signal, at wavelengths 380 nm-450 nm. SHG signal is generated in nano-scale volumes using scanned, femto-second pulsed tunable lasers.

Initial experiments have focused on construction of the microscope module for dynamic focal screening (DFS), demonstration of photoconjugation, and simulation of development of birefringence using simple closed-loop feedback (FIGS. 10A-10D).

FIGS. 10A-10D show a prototype dynamic focal screening platform consisting of 9×16 multiplexed light emitting diode (LED) array (#2973, Adafruit, Inc.), a 320×480 pixel thin-film transistor liquid crystal display (TFT-LCD, #2050, Adafruit, Inc.), and microcontroller (UnoRev3, Arduino) on a microscope base (MT9930, Meiji, Inc.). Blue light patterns are projected onto a microscope image plane (FIG. 10B) from the setup, controllable through MATLAB. FIG. 10C shows fluorescence signal from collagen after conjugation of green fluorescent protein (GFP) to the collagen network using a Ruthenium crosslinker (XL), blue light exposure, and extensive rinsing (hv+rinse). FIG. 10D shows a raw birefringence image of a collagen network aligned circumferentially around an emplaced bubble, with idealized, tessellated birefringence map (ref). Simulated feedback transforms a random field (start) to a birefringence map approaching the simulated image over 1 (loop 1) to 10 cycles (loop 10), with a structural similarity index>0.9 after 10 cycles, depending on the rate of error correction (graph of SSI versus loop number).

The Dynamic focal screening (DFS) module should work on most microscopes, based on images of any modality (fluorescence, phase contrast, differential interference contrast, brightfield, etc.). The LED array can be switched from blue LEDs (450 nm) to any other wavelength or alternatively, can be any light source (laser, mercury arc lamp, halogen) designed to create a uniform illumination field or interferometry set-up behind a digitally-controlled screen, or alternatively, any scanned focused laser source. The tissue construct can be any hydrogel material, with any embedded cells, but with requirements of reasonably transparency and thickness <1 mm to avoid diffusion of light patterns and allow focusing of light patterns using extra long-working distance objectives. The reagent kit can contain any kind of photocrosslinker, including ruthenium-based, and photoinitiators triggered by ultraviolet, visible, or infrared light. Similarly, any bioactive molecules can be included, such as growth factors, chemokines, and bioactive peptides.

Bioprinting is a subset of biofabrication in which custom tissue structures and spatial distributions of multiple cell types are created by layering and/or spatially-directed emplacement of multiple bioinks, which are cell-laden hydrogels (14). Photocrosslinking enhances the mechanical properties and shape retention of extrusion-printed methacrylated collagen (15), gelatin (16), hyaluronic acid (17), and various composites (18, 19). Alternatively, the recently demonstrated FRESH technique uses supportive gelatin microspheres to retain the shape of printed collagen structures, which require extra support during collagen network and fibril self-assembly (20). Still, most hydrogel constructs lack the microstructural complexity and mechanical properties of native tissue (21). Spatial patterning through biofabrication, including bioprinting, partially addresses tissue construct microstructural and mechanical deficiencies.

Crosslinking of tissue scaffolds is a viable approach to improve construct mechanics, stability during culture, and biomechanical functionality. Chemical crosslinkers such as glutaraldehyde are highly toxic to cells in their reactive state but were successfully employed in cardiac valve and other implants (22, 23). Protein crosslinking typically modifies the amino acids tyrosine or lysine through phenol and epsilon amine group reactivity, respectively, to form covalent intrachain bonds leading to a higher bulk stiffness of crosslinked scaffolds (24, 25). Crosslinking enzymes resident in tissues include transglutaminase (26) and lysyl oxidase (27) and were used successfully to crosslink hydrogel tissue constructs (28-30). Enzyme activity is difficult to control spatially, so crosslinking enzymes have not been used extensively in bioprinting. In comparison, photocrosslinking reagents for use in bioprinting may be activated in a spatially selective manner, using photomasks (31), laser scanning (32-34), or digital light processing (35) to create crosslink patterns in tissue constructs. Many photocrosslinkers are most efficiently activated by ultraviolet light, which is phototoxic to most cells. In contrast, blue light activation of ruthenium and persulfate leads to dityrosine crosslinks in hydrogels with minimal toxicity (12, 36, 37).

In native tissues, spatially heterogenous crosslink density and mechanical properties are relevant to tissue function, and in cancer and aging. Crosslinks play a vital role in the normal mechanical functioning of skin, articular cartilage, tendons (38), ligaments, bones, and other connective tissues. For example, crosslinking stabilizes corneal shape and refractive properties, and is used to treat keratoconus and correct blurred vision (15). In breast cancer, local collagen density and crosslinking are associated with a greater risk of metastases (13, 14). Local tumor stroma stiffness also influences the mode and rate of breast cancer cell invasion. Higher stiffness of tissues over years, as seen in aging, contributes to pathologies including osteoarthritis, diabetes, and atherosclerosis (12).

Disclosed embodiments may create photocrosslink patterns in tissue constructs using an inexpensive and flexible platform that enables concurrent imaging of patterns and subsequent cell and tissue alterations, with the option for subsequent, additional photocrosslinking. The rationale for such a platform is to mimic the spatiotemporal control of crosslinking by cells in native tissues. Aims are designed to test the hypotheses that visible light photocrosslink patterns emplaced in tissue constructs 1) alter local protein network density, 2) guide cell movement and alignment, and 3) conjugate soluble proteins to the solid biomacromolecular network. First, an inexpensive inverted microscope for teaching and home use may be modified to allow patterned photocrosslinking with a blue light LED array, a projection system, and an optional digitally controlled liquid crystal display mask. Then, photocrosslinking patterning is demonstrated to produce contrast in brightfield and phase contrast microscopy, with contrast changes and wet weight reduction consistent with crosslink-induced hydrogel deswelling. Photocrosslinking also conjugates soluble green fluorescent protein to gelatin and collagen hydrogels. Finally, cells respond to local crosslink emplacement by aligning toward regions of higher crosslink density. Small focal defects may be created in collagen hydrogels using a crosslinking method that produces solid stress in the collagen network that exceeded the local network failure strength. The results demonstrate the feasibility of inexpensive and flexible spatiotemporal patterning of photocrosslinks in tissue constructs activated by blue light, allowing for image-guided patterning of biofabricated and engineered tissues during culture.

Patterned photocrosslinking has several uses in the biofabrication of microstructurally complex tissue constructs, through both photolithography of scaffolds and photoconjugation of cell adhesive and instructive moieties. Often the polymers used are modified by methacrylation while photoactivation requires ultraviolet light. In contrast, disclosed embodiments aim to design, build and evaluate a low-cost platform to place photocrosslink patterns into unmodified collagen and gelatin hydrogels using visible light and ruthenium-mediated tyrosine crosslinking in a way compatible with cell culture and inverted microscopes commonly used in biological laboratories. In some disclosed embodiments, a photoprinting module is constructed above an inverted microscope sample stage to be confocal with the imaging system. The module consists of a blue light emitting diode array, light pipe, diffuser, microelectronically controlled liquid crystal display as photomask, and focusing objective. Resulting Ruthenium-mediated photocrosslink patterns were visible in unmodified collagen and gelatin hydrogels due to altered local polymer network density and optical contrast. Green fluorescent protein was conjugated in patterns to both gelatin and collagen gels, dependent on light exposure, intensity, and polymer network density. Pattern resolution varied from 2.0±0.5 μm to 102±33 μm dependent on the focusing objective magnification and the pattern used (LCD pixel versus LED element). Further, photocrosslink patterns placed in collagen hydrogels and incubated without rinsing in serum-containing media swelled over 20-48 hours, breaking the collagen network and forming approximately ˜50 μm diameter holes. Fibroblasts cultured in photopatterned collagen hydrogels aligned and moved on and around crosslinked regions, consistent with durotaxis and contact guidance. The platform for disclosed photocrosslinking will impact several research fields, notably bioprinting of microstructurally and mechanically complex tissue constructs.

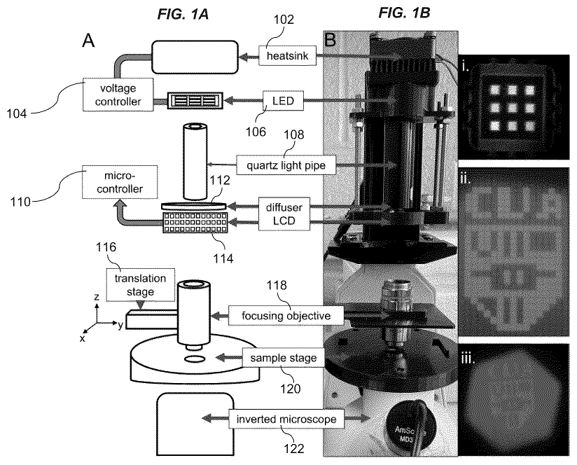

FIGS. 1A-1B illustrate schematic and photographs of a disclosed photocrosslink patterning device, including (i) a light emitting diode array, (ii) a graphic LCD screen, and (iii) a blue light pattern imaged by the microscope, according to one embodiment of the present disclosure.

Embodiments of the disclosed device (FIGS. 1A-1B) may include a heatsink (102), voltage controller (104), LED (106), quartz light pipe (108), micro-controller (110), diffuser (112), LCD (114), translation stage (116), focusing objective (118), sample stage (120), and inverted microscope (122).

### Results

Gelatin Crosslinking with LCD Creates High Resolution Patterns

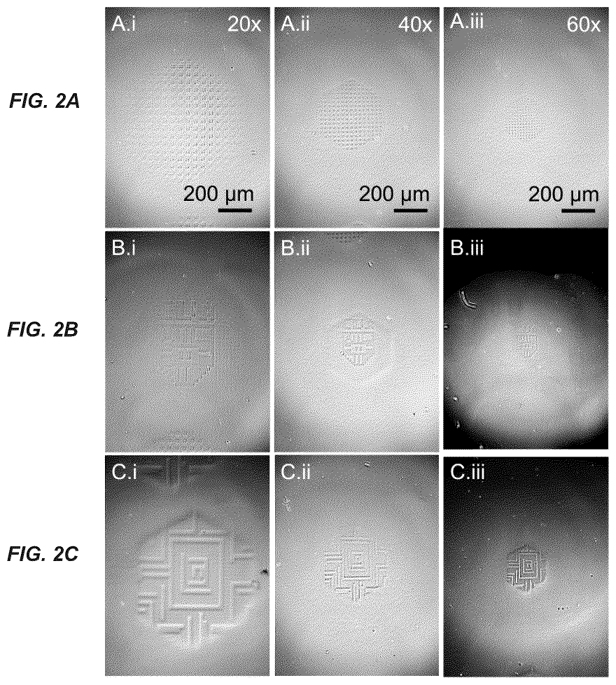

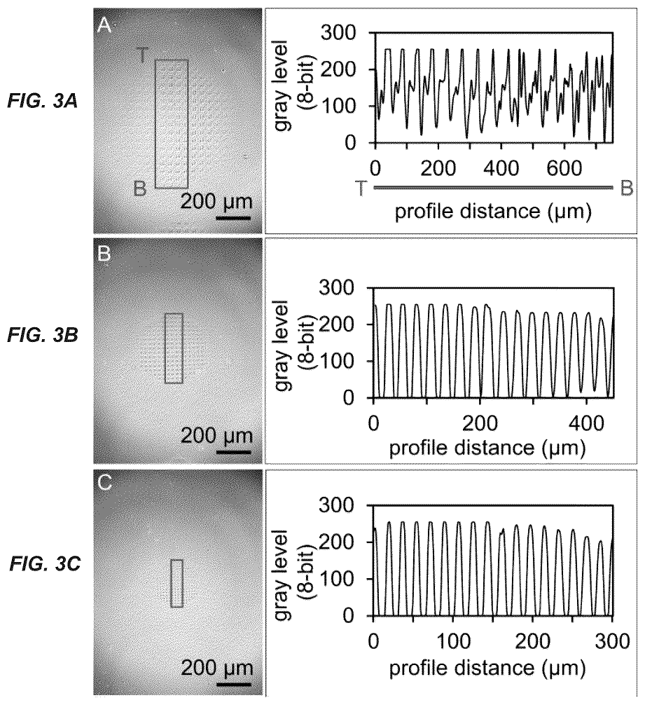

In gelatin, blue light patterns produced by an LCD mask may produce digitally controlled Ruthenium-mediated crosslink patterns visible through a phase contrast microscope (FIGS. 2A-2C). The magnification factor of the projecting microscope lens may minify the LCD pattern by the same amount with no loss of pattern detail. Compared to the patterns projected through a 20× objective (FIGS. 2A-2C.i), the 40× and 60× objectives produced patterns 2- and 3-fold finer, over a smaller area. The circumscribing hexagon visible in the patterns resulted from background emanating from the similarly shaped light pipe, through the dark areas of the LCD, which had a contrast ratio of 5. Profiles were plotted through the center of the crosslinked dot patterns (FIGS. 3A-3C). The full-width half-max (FWHM) of single pixel printed features are approximately 27.1±3.9 μm, 11.8±1.1 μm. 8.7±0.5 μm, and 2.0±0.5 μm for 20×, 40×, 60×, and 100× objectives, respectively (mean±standard deviation). The features are relatively uniform across the hexagonal illumination, which was slightly smaller than the LCD itself. The more variable feature boundaries at 20× result from phase contrast edge artefacts that occur across contrast boundaries at the scale of features printed with the 20× objective and are notably absent at the higher minification factors of 40× and 60×.

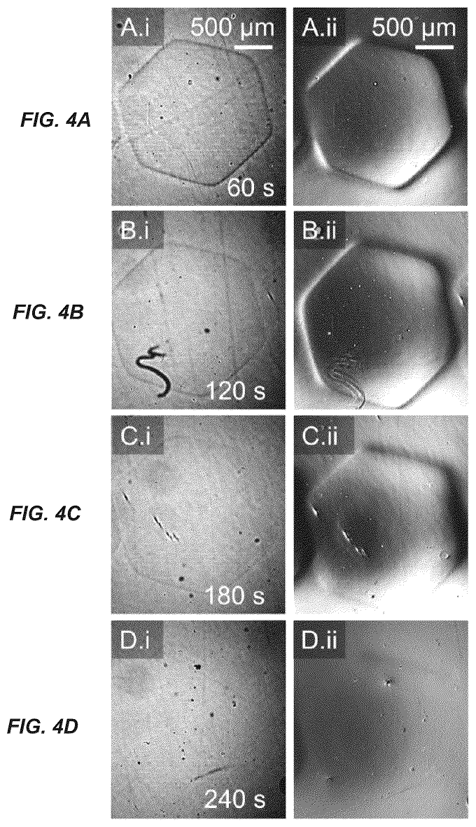

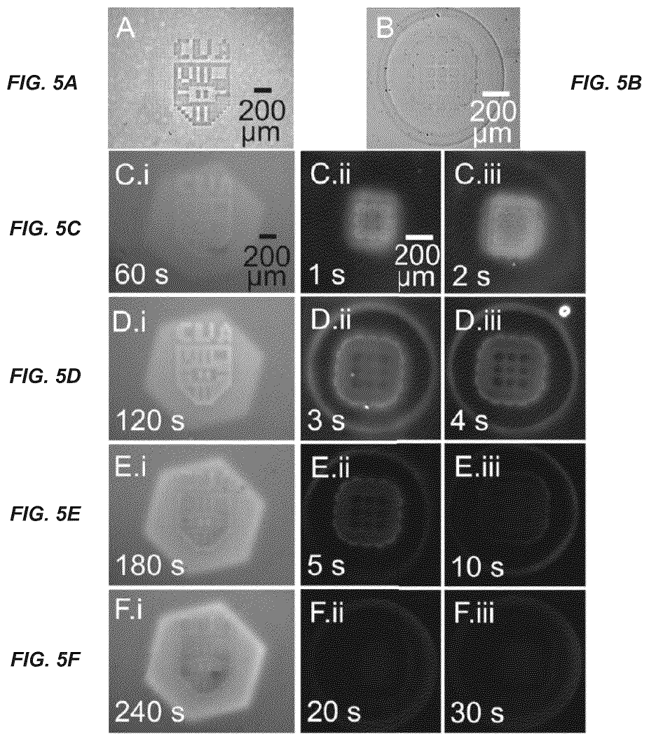

In collagen hydrogels, blue light patterns produce only faint crosslink-related contrast visible in brightfield and phase contrast micrographs (FIGS. 4A-4D). Moreover, these patterns appear most distinctly after 60 seconds exposure, with reduced contrast at longer exposure, up to 240) seconds. At the same time, the contrast between regions illuminated by light pixels and, through background illumination and scattering, dark pixels, are also poor compared to the contrast of patterns in gelatin. Photopatterned contrast in collagen gels become higher with the LCD removed and the LED array sending light directly through the objective and into the gel (FIG. 5B). Both LCD-projected light in gelatin and LED-projected light in collagen conjugate GFP to the hydrogel polymer network, producing fluorescent patterns of protein visible by microscopy (FIGS. 5A-5F). In gelatin, 120 seconds of exposure produce the highest contrast of signal to background, with progressive loss of patterned signal at longer exposures (FIGS. 5C.i-5F.i). Notably, the GFP signal-to-background ratio of the LED image in collagen is highest at 1 second of blue light exposure, with longer exposures leading to lower signal from the LED spots, and progressively higher signal from reflective portions of the LED chip, up to 30 seconds (FIGS. 5C-5F.ii-.iii).

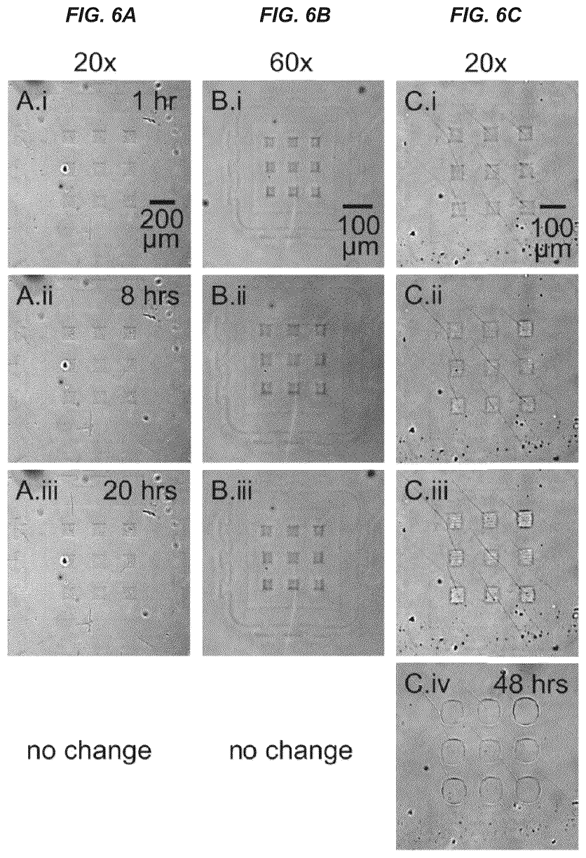

Collagen photopatterns produced by the LED array rival the resolution of gelatin photopatterns produced by the LCD. At 20× and 60× minification, photoelements that were 1×1 (mm×mm) on the array were 102.0±33.2 μm and 38±3 μm in the collagen gel (FIG. 6A vs. FIG. 6B, respectively). Further, at 20× minification, the 5 narrow rectangular elements of each LED formed visible features in the gel, with short-axis FWHM of 20.4±6.7 μm. While photopatterns in PBS-rinsed collagen gels remained stable over 48 hours of subsequent incubation, photopatterned collagen structures transferred to serum-containing medium without rinsing visibly swelled (FIG. 6C.i-iv), breaking the hydrogel and forming round defects ˜50 μm in diameter between 20-48 hours after changing the medium.

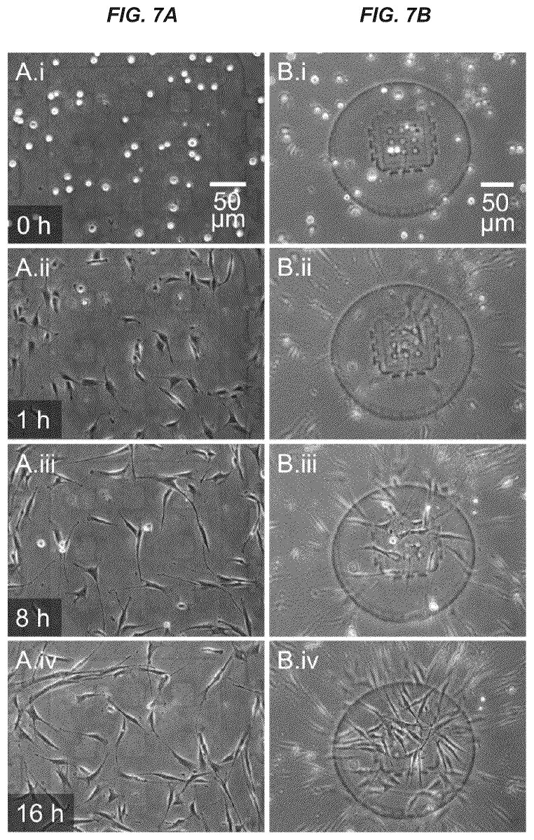

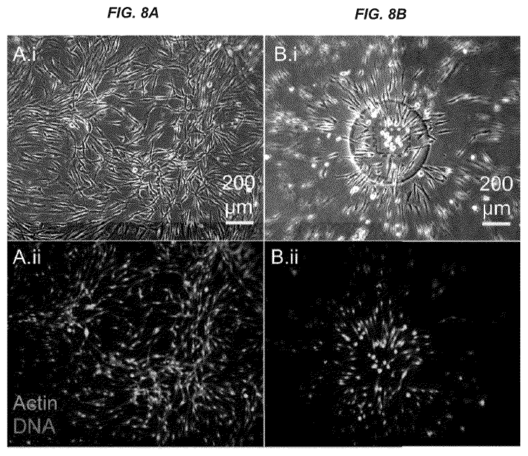

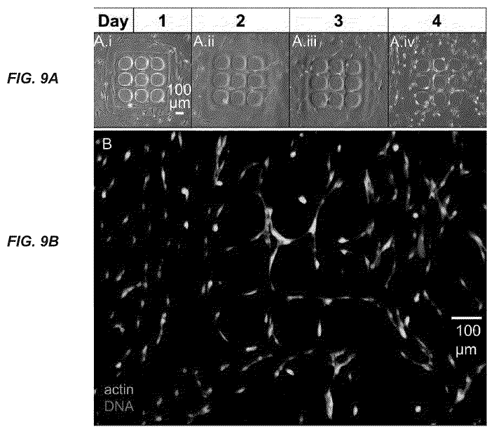

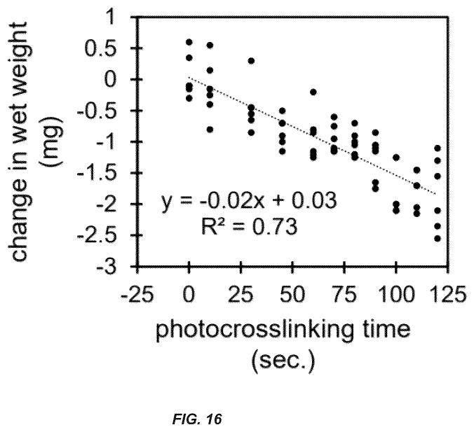

Unswollen collagen photocrosslink patterns cause fibroblast alignment (FIGS. 7A-7B). Larger structures printed at 20× minification caused fibroblasts to align with long axes parallel to local photocrosslink feature edges (FIGS. 7A.i-7A.iv), about 18 hours after adding the cells. In comparison, the much smaller photocrosslink features printed at 60× minification caused fibroblasts to align radially toward the circular feature defining the outer limit of photocrosslinking, and at a more random orientation within the crosslinked image of the LED array (FIGS. 7B.i-7B.iv). Staining of cell nuclei and the F-actin cytoskeleton accentuated the differences in alignment and orientation of fibroblasts across larger and smaller crosslink patterns (FIGS. 8A-8B). In contrast, cells aligned circumferentially around circular defects created by crosslinked gel swelling (FIGS. 9A-9B). Importantly, gelatin plugs lost wet weight upon photocrosslinking, −0.016±0.001 mg/second (mean±standard error, Student's t-test, t=−13.2, p<0.001) for up to 120 seconds (ANOVA, R²=0.73, F=175, p<0.001; FIG. 16). The linear best-fit intercept is not significantly different from 0 (Student's t-test, p=0.73). Gelatin plugs exposed to blue light for 90 seconds without added crosslinking reagents have no significant change in wet weight (mean±SD, 0.05±0.3 mg, no difference pre- vs. post-light exposure, Student's paired t-test, p=0.74).

In one embodiment, an apparatus may comprise an inverted microscope sample stage; a photomask; a light diffuser; a light pipe; and a light source, wherein the light source is confocal with the inverted microscope, and wherein the apparatus is configured to produce a ruthenium-mediated photocrosslink pattern into a subject.

In some embodiments, the photomask is disposed between the inverted microscope sample stage and the light diffuser, the light diffuser is disposed between the photomask and the light pipe, the light pipe is disposed between the light diffuser and the light source, and the light source is disposed above the light pipe.

In one embodiment, the photomask is a monochrome LCD.

In one embodiment, the light pipe is a quartz light pipe.

In one embodiment, the light source is selected from the group consisting of a laser, a mercury arc lamp, an LED, and a halogen.

In one embodiment, the light source is the LED.

In one embodiment, the subject comprises a hydrogel.

In one embodiment, the subject comprises a group consisting of a collagen hydrogel and a gelatin hydrogel.

In one embodiment, the photocrosslink pattern is placed in the collagen hydrogel, incubated and swelled over 15 to 55 hours forming 35 to 70 μm diameter holes.

In one embodiment, the gelatin hydrogel swells more in the presence of phosphate-buffered saline but swells less after crosslinking.

In one embodiment, a method of bioprinting comprises bioprinting a photocrosslink pattern to a subject using an apparatus comprising an inverted microscope sample stage; a photomask; a light diffuser; a light pipe; and a light source, wherein the light source is confocal with an inverted microscope, and wherein the apparatus produces a ruthenium-mediated photocrosslink pattern into the subject.

In one embodiment, the method modifies a protein network density and an optical contrast.

In one embodiment, the method conjugates a protein to a biomacromolecular network.

In one embodiment, the protein is a green fluorescent protein.

In one embodiment, the photocrosslink patterned subject is implemented in at least one of a medical tool kit, a fuel cell, a solar cell, an electronic cell, regenerative medicine and tissue regeneration, an implantable scaffold, a disease model, wound healing, 2D and 3D synthetic cell culture substrate, stem cell therapy, injectable therapies, biosensor development, high-throughput screening, biofunctionalized surfaces, printing biofabrication, and gene therapy.

In one embodiment, a method of wound treatment comprises bioprinting a photocrosslink pattern to a subject using an apparatus comprising: an inverted microscope sample stage; a photomask; a light diffuser; a light pipe; and a light source, wherein the light source is confocal with an inverted microscope, wherein the apparatus produces a ruthenium-mediated photocrosslink pattern into the subject, and applying the photocrosslink patterned subject to an individual.

In one embodiment, the individual is selected from the group consisting of a mammal, a reptile, a bird, a fish, an amphibian, and an invertebrate.

In one embodiment, the individual is a human.

In one embodiment, a kit comprises an effective amount of a material, wherein the material comprises: a photocrosslink patterned subject produced by an apparatus comprising: an inverted microscope sample stage; a photomask; a light diffuser; a light pipe; and a light source, wherein the light source is confocal with an inverted microscope, and wherein the apparatus is configured to produce a ruthenium-mediated photocrosslink pattern into the subject.

In one embodiment, a wound dressing or wound healing agent comprises a photocrosslink patterned subject produced by an apparatus comprising: an inverted microscope sample stage; a photomask; a light diffuser; a light pipe; and a light source, wherein the light source is confocal with an inverted microscope, and wherein the apparatus is configured to produce a ruthenium-mediated photocrosslink pattern into the subject.

In one embodiment, a pharmaceutical composition comprises a photocrosslink patterned subject produced by an apparatus comprising: an inverted microscope sample stage; a photomask; a light diffuser; a light pipe; and a light source, wherein the light source is confocal with an inverted microscope, and wherein the apparatus is configured to produce a ruthenium-mediated photocrosslink pattern into the subject.

In one embodiment, a device for delivering a photocrosslink patterned subject to an individual, wherein the photocrosslink patterned subject is produced by an apparatus comprises: an inverted microscope sample stage, a photomask, a light diffuser, a light pipe, and a light source, wherein the light source is confocal with an inverted microscope, and wherein the apparatus is configured to produce a ruthenium-mediated photocrosslink pattern into the subject.

### Discussion

In accordance with disclosed embodiments, an inexpensive and simple transmissive digital light projection platform may create microscale crosslink patterns in unmodified gelatin and type I collagen hydrogels. Pattern resolution may be controlled by the objective magnification used to project the image onto the gel surface, and by the pixel size of the LCD used to make the pattern. Pattern contrast may depend on light intensity, exposure time, LCD contrast ratio, and the type of material (collagen vs. gelatin), including material density. More intense light from the LED array, unfiltered by the LCD mask, may produce a high contrast pattern in 0.4% wt/vol collagen, whereas the LCD screen produced effective contrast in 3.5% wt/vol gelatin. Crosslink patterns visible by brightfield and phase contrast microscopy may be mirrored in fluorescence signal by photoconjugation of soluble GFP to gelatin and collagen, clearest in gelatin after 60 seconds light exposure, but in collagen after 1 second of more intense direct LED illumination. Crosslink patterns in collagen may swell over 48 hours to the point of breaking the hydrogel network when the gels are incubated with culture media without rinsing yet remain stable if rinsed. Adherent cells aligned with crosslink pattern features dependent on the feature size and shape. Wet weight measurements of photocrosslinked gelatin confirmed these gels lost wet weight, more with longer exposure to blue light. The properties and design of the current platform and future modifications will impact biofabrication approaches that use digital light projection and photolithography, while the reported findings are of significant value to guide the design of mechanically heterogenous, complex hydrogel tissue constructs.

The disclosed digital light transmission platform is inexpensive, flexible, and allows rapid pattern alteration, while selection of design features will improve resolution and contrast. The cost of objective, graphic LCD, light pipe, LED array, PCB, heat sink, fan, and 3-D printed parts was estimated to be approximately $2300. Typical photolithography instruments are more than an order of magnitude more expensive. Further, the setup can be installed on any inverted microscope base. With a mirror angled at 45°, the parts above the light-projecting objective can be arranged horizontally off the microscope light axis, preserving the supra-stage condenser present on most inverted microscopes. The light source, method of illumination diffusion, LCD and objective are all easily replaceable or switchable, for a variety of wavelengths, illumination patterns, and minification levels for multiplexing wavelength-dependent photoreactions in the same specimen. This allows several photocrosslinking and optogenetic reactions in the same field of view, or serial photoreactions with rinsing. Improvements to the current design would be to replace the graphic LCD with a high contrast thin film transistor-LCD, to improve power transmission to the projecting optics, and to add specimen scanning for continuous or repeated mask patterning. Importantly, because the optical mask and light projection device is confocal with an inverted microscope, the microscope image can be processed as input to the digital mask pattern, introducing feedback from the specimen into the photocrosslink writing process.

Emplaced photocrosslinks lead to local gel deswelling and optical contrast, while the unrinsed collagen gel clearly swells and breaks in the presence of serum-containing nutrient media and activated photoinitiators. This complex behavior requires further investigation. While the mechanism of deswelling is beyond the scope of this discussion, the phenomenon was previously observed in Ruthenium-crosslinked methacrylated gelatin (personal communication, Advanced Biomatrix, Inc.) and methacrylated gelatin-collagen photo-printed matrices (39). Gelatin hydrogels swell in the presence of phosphate-buffered saline but swell less after enhanced crosslinking: 241% over 24 hours for unmodified porcine gelatin, but only 10% after derivatization with Bolton-Hunter reagent producing higher crosslink content (40)). The Flory-Rehner treatment of equilibrium swelling theory relates crosslinked hydrogel swelling to the change in free energy of the hydrogel, dependent on the entropy of solvent-polymer mixing, heat of polymer-solvent mixing, and entropy change associated with the conformation of polymer segments under elastic tension (41). A more positive entropy of mixing moves the system toward greater swelling, whereas a more negative entropy of the polymer network favors less swelling, and possibly deswelling. Consistent with local crosslink density differences leading to spatially dependent swelling changes in the hydrogel, gelatin wrinkling around the crosslinked pattern was observed in early photolithography trials (data not shown), consistent with local hydrogel shrinkage. In collagen not rinsed with PBS, crosslink patterns of the LED spots clearly swelled, grew brighter in phase contrast, and then broke the collagen network leading to circular holes. Although collagen and gelatin contain 0.9% tyrosine, only about 11% of tyrosines form initial dityrosine bonds (40). Continued crosslinking of soluble proteins from nutrient media onto the insoluble scaffold might make both entropy terms more positive, contributing to this observed swelling. Other collagen and gelatin structures crosslinked after Ruthenium photoactivation displayed ˜1.5× swelling with subsequent structural stabilization (42).

Photo-conjugation of soluble GFP to gelatin and collagen demonstrates that this photolithography technique allows for the creation of biochemically complex patterned hydrogel constructs. Other photolithography approaches have also demonstrated patterned conjugation of soluble proteins and peptides to collagen-glycosaminoglycan (GAG) (43) and polyethylene-diacrylate (PEG-DA) (44) hydrogels. One advantage of the present technique is the ability to rapidly alter the photomask through digital control of the LCD screen. Thus, the photomask may be adapted to hydrogel structures and cell organization visible in the microscope field-of-view, so the emplacement of conjugated molecules depends on local tissue construct features. A simple motorized lateral translation system would allow large-scale photoconjugation across multiple fields-of-view. Future studies including liquid chromatography and mass spectrometry would reveal the stoichiometry of soluble protein conjugation to the hydrogel network. It is notable that the more intense LED image in collagen was apparent with a lower photoactivation time of 1-5 seconds than using the less intense LCD mask projection in gelatin, requiring 120 seconds. However, the LED spots themselves disappeared after 2 seconds of exposure, indicative of rapid photobleaching from the high light intensity. Therefore, optimal photocrosslinking of a soluble protein to a polymer network using Ruthenium depends on the local tyrosine content (dependent on polymer mass density), soluble protein concentration, photoactivation time and illumination intensity. The nature of bleaching or quenching of photoconjugated fluorescent proteins also requires further investigation.

Photocrosslink patterns in collagen and gelatin template the orientation of fibroblasts by alterations in topography, microstructural alignment, and potentially through stiffness gradients. The large and small LED chip patterns in collagen produced different effects on adherent fibroblasts (FIGS. 7A-7B and 8A-8B): limited alignment across spatial features several hundreds of microns across, but radial alignment toward the sub-50 μm crosslinked pattern. This radial alignment is consistent with durotaxis, in which motile cells move up a stiffness gradient (45). In contrast, fibroblasts align circumferentially around circular defects in collagen, consistent with contact guidance by underlying circumferential microstructural alignment (46).

In conclusion, photocrosslink patterning with visible light projected through a digitally controlled LCD array produces microscale crosslink patterns in unmodified natural biopolymer hydrogels with facile tuning of contrast, crosslink density, and photoconjugated soluble proteins. Furthermore, the confocal design of photopatterning and microscopy allows incorporation of microscope image features into the photomask design. Future work will improve the photomask resolution, contrast, and bit-depth to create smooth crosslink gradients over a wider range of crosslink densities and will multiplex photoconjugated proteins in the same region. Prevascularized tissue constructs, and other spatially complex engineered tissues will benefit from biofabrication strategies incorporating photocrosslinking through digital masks. It is also anticipated that the platform described in this study, operated at several wavelengths and with repeated refreshment and rinsing of Ruthenium photocrosslinking reagents, enables serial photolithography and optogenetic manipulation of cellularized hydrogel constructs over the entire course of culture.

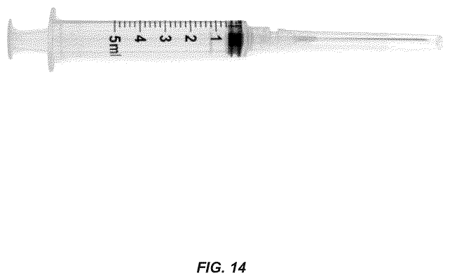

One of ordinary skills in the art would readily appreciate that any kind of device suitable for delivering the disclosed products described in the present disclosure may be utilized. For example, FIG. 14 illustrates an injectable device for delivering a photocrosslink patterned subject, according to one embodiment of the present disclosure. While the injectable device of FIG. 14 is illustrated as a syringe-type device, it is readily appreciated that the injectable device (FIG. 14) may not be limited to simply a syringe-type device. One of the ordinary skills in the art will readily appreciate that any injectable device suitable for delivering the photocrosslink patterned subject may be utilized according to aspects of the present disclosure.

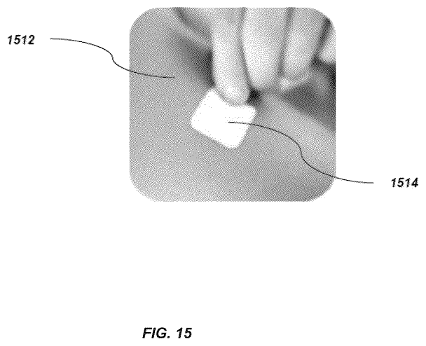

FIG. 15 is an illustrative representation of an exemplary delivery of the photocrosslink patterned subject into an individual's body such as through a patch. One of ordinary skills in the art will readily appreciate that any kind of patch suitable as a delivery device for delivering the disclosed products may be utilized. FIG. 15 illustrates patch 1514 comprising a photocrosslink patterned subject disclosed herein. Patch 1514 may comprise an adhesive patch for placing on the skin or surfaces 1512 of an individual and one or more active layers embedded in the adhesive patch, wherein the one or more active layers comprise a photocrosslink patterned subject disclosed herein in a therapeutically effective amount. Patch 1514 may be placed on to an individual's body to allow the photocrosslink patterned subject embedded in the adhesive patch to be delivered into the individual's bloodstream.

Having described the many embodiments of the present disclosure in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

### Examples

### Methods

### Photopatterning Instrument

The photopatterning device consists of the base of an inexpensive inverted microscope for teaching (IQCREW, Inverted microscope), a separate 10 W, 3×3 LED light source (MPJA, 32619), a quartz light pipe (Edmund Optics, N-BK7), light diffuser plate (Amscope), and a monochrome LCD (Crystal Fontz, CFAO4265ATTL) (FIGS. 1A-1B). The illuminator and condenser arm above the sample stage may be removed. A metallurgical or epifluorescence microscope with no parts above the sample stage, any inverted microscope with a hinged neck, or optical components in a cage system or on an optical bench are feasible alternative designs. In the device used for this study, all optical component holders and structural parts may be designed with CAD software (Solidworks) and parts may be printed in PLA using a 3D printer (Creality, CR10V3). The LED array consists of a 3×3 square pattern of individual ˜1 Watt diodes emitting blue light centered at 430 nm. The LED array is powered by a separate controllable digital buck converter and appropriate LED Driver. The separation between the front of the LED and the light pipe was <2 mm. The light pipe, diffuser, and LCD are removable. Just above the sample stage, a selected microscope objective projects a minified image of the LCD or LED. Extra-long working distance (ELWD) objectives are used at magnifications of 20×, 40×, 60×, and 100×. The objectives are housed in a completely 3D printed holder with X, Y, and Z linear displacement to aid in light path alignment. For calibration and monitoring the microscope was also fitted with a CMOS camera (Amscope, MD300).

### Alignment of the Microscope and LCD Projection

The microscope and LCD setup may be aligned and calibrated by first focusing on a slide sample with a cover slip that was marked on the contacting side of the coverslip and microscope slide and using the CMOS camera to verify proper focus. The LCD may then be focused by using the second focusing objective and a three-axis translation stage to focus the LCD pattern onto the already focused slide and coverslip sample.

### Photocrosslinking and Specimen Preparation

A ruthenium and sodium persulfate crosslinking kit may be used (Advanced Biomatrix, 5248). Work concentrations were 2× manufacturer recommendations. Gelatin hydrogels may be prepared using food-grade bovine gelatin powder and 1×PBS to a total concentration of 35 mg/ml. Ruthenium and sodium persulfate may be added to the gelatin hydrogel to a concentration of 4 μL/mL. Gelatin gel final height in 35 mm-diameter Petri dishes was 1 mm. When gelatin is poured on top of glass slides with a coverslip on top, the final gel height is 0.5 mm. The gels may be allowed to form at room temperature and covered and preserved for 2 to 24 hours, depending on sample volume, before patterning.

### Collagen Preparation

Collagen hydrogels may be made at 4 mg/ml with ruthenium and sodium persulfate added the solution to have a concentration of 4 μL/mL. The solution may then be added to a 35 mm-diameter Petri dish to a height of 0.5 mm. Collagen gels on slides may be prepared using the same method as with gelatin, to a 0.5 mm height. The gel may then be patterned either within 15 minutes, before self-assembly is complete, or after 1-2 hours, when self-assembly is complete. Collagen gels may be incubated at 37° C. for 30 minutes to complete self-assembly, and then rinsed with 1×PBS or incubated in nutrient media without rinsing, depending on the experiment. The gels may be imaged after photopatterning.

### Photocrosslink Pattern Formation

Patterns made using the LCD may be created by using a microcontroller (Uno Rev3, Arduino) with code to run different bitmaps. The patterns may then be projected and focused onto the gelatin and collagen gel surfaces using different focusing objectives depending on desired pattern size. The exposure times may be controlled using a timer to control LED emission ranging from intervals of 1 second to 6 minutes. The LED voltage may be set at 9.34 volts. For patterns utilizing the LED only (without the quartz light pipe, diffuser, and LCD) the voltage may be set to 12 V by using the buck converter and LED driver. The pattern of the LED may then be focused onto the sample surface with the focusing objective with the same range of exposure times as the LCD pattern.

### Patterned Collagen GFP Photoconjugation

Collagen hydrogels may be made to a concentration of 6 mg/ml using the same concentrations for ruthenium and sodium persulfate as before. GFP may then be added to the collagen solution diluting from the stock concentration by a factor of 3. The working concentration of collagen is approximately 4 mg/ml. The material may then be added to a glass slide or Petri dish and a circular coverslip added on top. The gel height is approximately 0.5 mm. Hydrogels may then be immediately photopatterned using the 40×ELWD objective. Exposure times ranged from 1-30 seconds. Gels may then be incubated for a further 30 minutes and then rinsed in 1×PBS for 30 minutes before imaging with epifluorescence microscopy.

### Cell Culture and Staining

Human gingival fibroblasts (HGFs) may be seeded onto photopatterned collagen hydrogels. Images may be collected during subsequent culture by phase contrast microscopy. Tissue constructs may then be cultured for 1-4 days before staining with a phalloidin conjugated to a fluorescent green dye (R37110, ThermoFisher Scientific) and propidium iodide (P1304MP, ThermoFisher Scientific). Specimens may then be imaged using epifluorescence microscopy.

### Time Lapse Imaging

Time lapse imaging over 20 hours may be conducted on patterned gels to evaluate photocrosslink pattern swelling characteristics, cell motility and alignment. An on-stage incubator (Ibidi) may be used to enable cell culture during time-lapse imaging.

### REFERENCES

The following references are referred to above and are incorporated herein by reference:
1. Wu L Q, Payne G F. Biofabrication: using biological materials and biocatalysts to construct nanostructured assemblies. Trends Biotechnol. 2004; 22(11): 593-9. Epub 2004 Oct. 20. doi: 10.1016/j.tibtech.2004.09.008. PubMed PMID: 15491804.
2. Pereira R F, Barrias C C, Granja P L, Bartolo P J. Advanced biofabrication strategies for skin regeneration and repair. Nanomedicine (Lond). 2013; 8(4):603-21. Epub 2013 Apr. 9. doi: 10.2217/nnm.13.50. PubMed PMID: 23560411.
3. Knowlton S, Yenilmez B, Tasoglu S. Towards Single-Step Biofabrication of Organs on a Chip via 3D Printing. Trends Biotechnol. 2016; 34(9):685-8. Epub 2016 Jul. 20. doi: 10.1016/j.tibtech.2016.06.005. PubMed PMID: 27424152.
4. Liu Y, Kim E, Ghodssi R, Rubloff G W, Culver J N, Bentley W E, Payne G F. Biofabrication to build the biology-device interface. Biofabrication. 2010; 2(2):022002. Epub 2010 Sep. 3. doi: 10.1088/1758-5082/2/2/022002. PubMed PMID: 20811128.
5. Stephens K, Zakaria F R, VanArsdale E, Payne G F, Bentley W E. Electronic signals are electrogenetically relayed to control cell growth and co-culture composition. Metab Eng Commun. 2021; 13:e00176. Epub 2021 Jul. 2. doi: 10.1016/j.mec.2021.e00176. PubMed PMID: 34194997; PMCID: PMC8233222.
6. Hu S, Chen T H, Zhao Y, Wang Z, Lam R H W. Protein-Substrate Adhesion in Microcontact Printing Regulates Cell Behavior. Langmuir. 2018; 34(4): 1750-9. Epub 2018 Jan. 7. doi: 10.1021/acs.langmuir.7b02935. PubMed PMID: 29304548.
7. Yang X, Mohseni M, Bas O, Meinert C, New E J, Castro N J. Type II Photoinitiator and Tuneable Poly(Ethylene Glycol)-Based Materials Library for Visible Light Photolithography. Tissue Eng Part A. 2020; 26(5-6): 292-304. Epub 2020 Jan. 8. doi: 10.1089/ten.TEA.2019.0282. PubMed PMID: 31910098.
8. Lee H J, Lee J S, Chansakul T, Yu C, Elisseeff J H, Yu S M. Collagen mimetic peptide-conjugated photopolymerizable PEG hydrogel. Biomaterials. 2006; 27(30):5268-76. Epub 2006 Jun. 27. doi: 10.1016/j.biomaterials.2006.06.001. PubMed PMID: 16797067.
9. Raub C B, Putnam A J, Tromberg B J, George S C. Predicting bulk mechanical properties of cellularized collagen gels using multiphoton microscopy. Acta Biomater. 2010; 6(12):4657-65. Epub 2010 Jul. 14. doi: 10.1016/j.actbio.2010.07.004. PubMed PMID: 20620246; PMCID: PMC3373188.
10. Liu C, Chiang B, Lewin Mejia D, Luker K E, Luker G D, Lee A. Mammary fibroblasts remodel fibrillar collagen microstructure in a biomimetic nanocomposite hydrogel. Acta Biomater. 2019; 83:221-32. Epub 2018 Nov. 11. doi: 10.1016/j.actbio.2018.11.010. PubMed PMID: 30414485; PMCID: PMC6291359.
11. Kwee B J, Mooney D J. Biomaterials for skeletal muscle tissue engineering. Curr Opin Biotechnol. 2017; 47:16-22. Epub 2017 Jun. 3. doi: 10.1016/j.copbio.2017.05.003. PubMed PMID: 28575733; PMCID: PMC5617779.
12. Ly K L, Luo X, Raub C B. Oral mucositis on a chip: modeling induction by chemo- and radiation treatments and recovery. Biofabrication. 2022; 15(1). Epub 2022 Sep. 21. doi: 10.1088/1758-5090/ac933b. PubMed PMID: 36126642.
13. Moroni L, Burdick J A, Highley C, Lee S J, Morimoto Y, Takeuchi S, Yoo J J. Biofabrication strategies for 3D in vitro models and regenerative medicine. Nat Rev Mater. 2018; 3(5):21-37. Epub 2019 Jun. 22. doi: 10.1038/s41578-018-0006-y. PubMed PMID: 31223488; PMCID: PMC6586020.
14. Matai I, Kaur G, Seyedsalehi A, McClinton A, Laurencin C T. Progress in 3D bioprinting technology for tissue/organ regenerative engineering. Biomaterials. 2020; 226:119536. Epub 2019 Oct. 28. doi: 10.1016/j.biomaterials.2019.119536. PubMed PMID: 31648135.
15. Brinkman W T, Nagapudi K, Thomas B S, Chaikof E L. Photo-cross-linking of type I collagen gels in the presence of smooth muscle cells: mechanical properties, cell viability, and function. Biomacromolecules. 2003; 4(4):890-5. Epub 2003 Jul. 15. doi: 10.1021/bm0257412. PubMed PMID: 12857069.
16. Chen Y C, Lin R Z, Qi H, Yang Y, Bae H, Melero-Martin J M, Khademhosseini A. Functional Human Vascular Network Generated in Photocrosslinkable Gelatin Methacrylate Hydrogels. Adv Funct Mater. 2012; 22(10):2027-39. Epub 2012 Aug. 22. doi: 10.1002/adfm.201101662. PubMed PMID: 22907987; PMCID: PMC3422083.
17. Baier Leach J, Bivens K A, Patrick C W, Jr., Schmidt C E. Photocrosslinked hyaluronic acid hydrogels: natural, biodegradable tissue engineering scaffolds. Biotechnol Bioeng. 2003; 82(5):578-89. Epub 2003 Mar. 26. doi: 10.1002/bit.10605. PubMed PMID: 12652481.
18. Moncal K K, Ozbolat V, Datta P, Heo D N, Ozbolat I T. Thermally-controlled extrusion-based bioprinting of collagen. J Mater Sci Mater Med. 2019; 30(5):55. Epub 2019 May 2. doi: 10.1007/s10856-019-6258-2. PubMed PMID: 31041538.
19. Rastogi P, Kandasubramanian B. Review of alginate-based hydrogel bioprinting for application in tissue engineering. Biofabrication. 2019; 11(4):042001. Epub 2019 Jul. 18. doi: 10.1088/1758-5090/ab331e. PubMed PMID: 31315105.
20. Lee A, Hudson A R, Shiwarski D J, Tashman J W, Hinton T J, Yerneni S, Bliley J M, Campbell P G, Feinberg A W. 3D bioprinting of collagen to rebuild components of the human heart. Science. 2019; 365(6452): 482-7. Epub 2019 Aug. 3. doi: 10.1126/science.aav9051. PubMed PMID: 31371612.
21. Antoine E E, Vlachos P P, Rylander M N. Review of collagen I hydrogels for bioengineered tissue microenvironments: characterization of mechanics, structure, and transport. Tissue Eng Part B Rev. 2014; 20(6):683-96. Epub 2014 Jun. 14. doi: 10.1089/ten.TEB.2014.0086. PubMed PMID: 24923709; PMCID: PMC4241868.
22. Schoen F J, Levy R J. Calcification of tissue heart valve substitutes: progress toward understanding and prevention. Ann Thorac Surg. 2005; 79(3): 1072-80. Epub 2005 Mar. 1. doi: 10.1016/j.athoracsur.2004.06.033. PubMed PMID: 15734452.
23. Krishnakumar G S, Sampath S, Muthusamy S, John M A. Importance of crosslinking strategies in designing smart biomaterials for bone tissue engineering: A systematic review. Mater Sci Eng C Mater Biol Appl. 2019; 96:941-54. Epub 2019 Jan. 5. doi: 10.1016/j.msec.2018.11.081. PubMed PMID: 30606606.
24. Wang X, Ao Q, Tian X, Fan J, Tong H, Hou W, Bai S. Gelatin-Based Hydrogels for Organ 3D Bioprinting. Polymers (Basel). 2017; 9(9). Epub 2017 Aug. 30. doi: 10.3390/polym9090401. PubMed PMID: 30965706; PMCID: PMC6418925.
25. Rico-Llanos G A, Borrego-Gonzalez S, Moncayo-Donoso M, Becerra J, Visser R. Collagen Type I Biomaterials as Scaffolds for Bone Tissue Engineering. Polymers (Basel). 2021; 13(4). Epub 2021 Mar. 7. doi: 10.3390/polym13040599. PubMed PMID: 33671329; PMCID: PMC7923188.
26. Iismaa S E, Mearns B M, Lorand L, Graham R M. Transglutaminases and disease: lessons from genetically engineered mouse models and inherited disorders. Physiol Rev. 2009; 89(3):991-1023. Epub 2009 Jul. 9. doi: 10.1152/physrev.00044.2008. PubMed PMID: 19584319.
27. Eyre D R, Paz M A, Gallop P M. Cross-linking in collagen and elastin. Annu Rev Biochem. 1984; 53:717-48. Epub 1984 Jan. 1. doi: 10.1146/annurev.bi.53.070184.003441. PubMed PMID: 6148038.
28. Tirella A, Mattei G, La Marca M, Ahluwalia A, Tirelli N. Functionalized Enzyme-Responsive Biomaterials to Model Tissue Stiffening in vitro. Front Bioeng Biotechnol. 2020; 8:208. Epub 2020 Apr. 24. doi: 10.3389/fbioe.2020.00208. PubMed PMID: 32322576; PMCID: PMC7156543.
29. Yung C W, Wu L Q, Tullman J A, Payne G F, Bentley W E, Barbari T A. Transglutaminase crosslinked gelatin as a tissue engineering scaffold. J Biomed Mater Res A. 2007; 83(4): 1039-46. Epub 2007 Jun. 23. doi: 10.1002/jbm.a.31431. PubMed PMID: 17584898.
30. Nair M, Calahorra Y, Kar-Narayan S, Best S M, Cameron R E. Self-assembly of collagen bundles and enhanced piezoelectricity induced by chemical crosslinking. Nanoscale. 2019; 11(32):15120-30. Epub 2019 Aug. 2. doi: 10.1039/c9nr04750f. PubMed PMID: 31369017; PMCID: PMC7745105.
31. Hwang H H, You S, Ma X, Kwe L, Victorine G, Lawrence N, Wan X, Shen H, Zhu W, Chen S. High throughput direct 3D bioprinting in multiwell plates. Biofabrication. 2021; 13(2). Epub 2020 Apr. 17. doi: 10.1088/1758-5090/ab89ca. PubMed PMID: 32299077.
32. Hribar K C, Meggs K, Liu J, Zhu W, Qu X, Chen S. Three-dimensional direct cell patterning in collagen hydrogels with near-infrared femtosecond laser. Sci Rep. 2015; 5:17203. Epub 2015 Nov. 26. doi: 10.1038/srep17203. PubMed PMID: 26603915; PMCID: PMC4658636.
33. Bardakova K N, Grebenik E A, Minaev N V, Churbanov S N, Moldagazyeva Z, Krupinov G E, Kostjuk S V, Timashev P S. Tailoring the collagen film structural properties via direct laser crosslinking of star-shaped polylactide for robust scaffold formation. Mater Sci Eng C Mater Biol Appl. 2020; 107:110300. Epub 2019 Nov. 26. doi: 10.1016/j.msec.2019.110300. PubMed PMID: 31761223.
34. Wang Z, Jin X, Tian Z, Menard F, Holzman J F, Kim K. A Novel, Well-Resolved Direct Laser Bioprinting System for Rapid Cell Encapsulation and Microwell Fabrication. Adv Healthc Mater. 2018; 7(9): e1701249. Epub 2018 Feb. 7. doi: 10.1002/adhm.201701249. PubMed PMID: 29405607.
35. Goodarzi Hosseinabadi H, Dogan E, Miri A K, Ionov L. Digital Light Processing Bioprinting Advances for Microtissue Models. ACS Biomater Sci Eng. 2022; 8(4): 1381-95. Epub 2022 Apr. 1. doi: 10.1021/acsbiomaterials. 1c01509. PubMed PMID: 35357144.
36. Bjork J W, Johnson S L, Tranquillo R T. Ruthenium-catalyzed photo cross-linking of fibrin-based engineered tissue. Biomaterials. 2011; 32(10):2479-88. Epub 2011 Jan. 5. doi: 10.1016/j.biomaterials.2010.12.010. PubMed PMID: 21196047; PMCID: PMC3791330.
37. Syedain Z H, Bjork J, Sando L, Tranquillo R T. Controlled compaction with ruthenium-catalyzed photochemical cross-linking of fibrin-based engineered connective tissue. Biomaterials. 2009; 30(35): 6695-701. Epub 2009 Sep. 29. doi: PMID: 19782397; PMCID: 10.1016/j.biomaterials.2009.08.039. PubMed PMC2853233.
38. Eekhoff J D, Fang F, Lake S P. Multiscale mechanical effects of native collagen cross-linking in tendon. Connect Tissue Res. 2018; 59(5):410-22. Epub 2018 Jun. 7. doi: 10.1080/03008207.2018.1449837. PubMed PMID: 29873266.
39. Lim K S, Schon B S, Mekhileri N V, Brown G C J, Chia C M, Prabakar S, Hooper G J, Woodfield T B F. New Visible-Light Photoinitiating System for Improved Print Fidelity in Gelatin-Based Bioinks. ACS Biomater Sci Eng. 2016; 2(10): 1752-62. Epub 2016 Oct. 10. doi: 10.1021/acsbiomaterials.6b00149. PubMed PMID: 33440473.
40. Elvin C M, Vuocolo T, Brownlee A G, Sando L, Huson M G, Liyou N E, Stockwell P R, Lyons R E, Kim M, Edwards G A, Johnson G, McFarland G A, Ramshaw J A, Werkmeister J A. A highly elastic tissue sealant based on photopolymerised gelatin. Biomaterials. 2010; 31(32):8323-31. Epub 2010 Aug. 3. doi: 10.1016/j.biomaterials.2010.07.032. PubMed PMID: 20674967.
41. Rehner J, Flory P J. Statistical Mechanics of Cross-Linked Polymer Networks II. Swelling. The Journal of Chemical Physics. 1943; 11:521-6. doi: doi.org/10.1063/1.1723792.
42. van der Sanden B, Gredy L, Wion D, Stephan O. 3D two-photon polymerization of smart cell gelatin-collagen matrixes with incorporated ruthenium complexes for the monitoring of local oxygen tensions. Acta Biomater. 2021; 130:172-82. Epub 2021 Jun. 16. doi: 10.1016/j.actbio.2021.06.021. PubMed PMID: 34129956.
43. Martin T A, Caliari S R, Williford P D, Harley B A, Bailey R C. The generation of biomolecular patterns in highly porous collagen-GAG scaffolds using direct photolithography. Biomaterials. 2011; 32(16):3949-57. Epub 2011 Mar. 15. doi: 10.1016/j.biomaterials.2011.02.018. PubMed PMID: 21397322; PMCID: PMC3947768.
44. Lee S H, Moon J J, West J L. Three-dimensional micropatterning of bioactive hydrogels via two-photon laser scanning photolithography for guided 3D cell migration. Biomaterials. 2008; 29(20):2962-8. Epub 2008 Apr. 25. doi: 10.1016/j.biomaterials.2008.04.004. PubMed PMID: 18433863: PMCID: PMC3942083.
45. Ebata H, Moriyama K, Kuboki T, Kidoaki S. General cellular durotaxis induced with cell-scale heterogeneity of matrix-elasticity. Biomaterials. 2020; 230:119647. Epub 2019 Dec. 4. doi: 10.1016/j.biomaterials.2019.119647. PubMed PMID: 31791844.
46. Lam V K, Phan T, Ly K, Luo X, Nehmetallah G, Raub C B. Dual-modality digital holographic and polarization microscope to quantify phase and birefringence signals in biospecimens with a complex microstructure. Biomed Opt Express. 2022; 13(2): 805-23. Epub 2022 Mar. 15. doi: 10.1364/BOE.449125. PubMed PMID: 35284161; PMCID: PMC8884236.

All documents, patents, journal articles and other materials cited in the present application are incorporated herein by reference.

While the present disclosure has been disclosed with references to certain embodiments, numerous modifications, alterations, and changes to the described embodiments are possible without departing from the sphere and scope of the present disclosure, as defined in the appended claims. Accordingly, it is intended that the present disclosure not be limited to the described embodiments, but that it has the full scope defined by the language of the following claims, and equivalents thereof.

What is claimed is:

1. An apparatus comprising:
an inverted microscope sample stage;
a photomask;
a light diffuser;
a light pipe; and
a light source,
wherein the light source is confocal with the inverted microscope, and
wherein the apparatus is configured to produce a ruthenium-mediated photocrosslink pattern into a subject.

2. The apparatus of claim 1,
wherein the photomask is disposed between the inverted microscope sample stage and the light diffuser,
wherein the light diffuser is disposed between the photomask and the light pipe,
wherein the light pipe is disposed between the light diffuser and the light source, and
wherein the light source is disposed above the light pipe.

3. The apparatus of claim 1, wherein the photomask is a monochrome LCD.

4. The apparatus of claim 1, wherein the light pipe is a quartz light pipe.

5. The apparatus of claim 1, wherein the light source is selected from the group consisting of a laser, a mercury arc lamp, an LED, and a halogen.

6. The apparatus of claim 5, wherein the light source is the LED.

7. The apparatus of claim 1, wherein the subject comprises a hydrogel.

8. The apparatus of claim 1, wherein the subject comprises a group consisting of a collagen hydrogel and a gelatin hydrogel.

9. The apparatus of claim 8, wherein the photocrosslink pattern is placed in the collagen hydrogel, incubated, and swelled over 15 to 55 hours forming 35 to 70 μm diameter holes.

10. The apparatus of claim 8, wherein the gelatin hydrogel swells more in the presence of phosphate-buffered saline but swells less after crosslinking.

11. A method of bioprinting comprising:
bioprinting a photocrosslink pattern to a subject using an apparatus comprising:
an inverted microscope sample stage;
a photomask;
a light diffuser;
a light pipe; and
a light source,
wherein the light source is confocal with an inverted microscope, and
wherein the apparatus produces a ruthenium-mediated photocrosslink pattern into the subject.

12. The method of claim 11,
wherein the photomask is disposed between the inverted microscope sample stage and the light diffuser,
wherein the light diffuser is disposed between the photomask and the light pipe,
wherein the light pipe is disposed between the light diffuser and the light source, and
wherein the light source is disposed above the light pipe.

13. The method of claim 11, wherein the light source is selected from the group consisting of a laser, a mercury arc lamp, an LED, and a halogen.

14. The method of claim 13, wherein the light source is the LED.

15. The method of claim 11 further comprising:
modifying a protein network density and an optical contrast.

16. The method of claim 11, wherein a protein is conjugated to a biomacromolecular network.

17. The method of claim 16, wherein the protein is a green fluorescent protein.

18. The method of claim 11, wherein the subject comprises a hydrogel.

19. The method of claim 11, wherein the subject comprises a group consisting of a collagen hydrogel and a gelatin hydrogel.

20. The method of claim 11, wherein the photocrosslink patterned subject is implemented in at least one of a medical tool kit, a fuel cell, a solar cell, an electronic cell, regenerative medicine and tissue regeneration, an implantable scaffold, a disease model, wound healing, 2D and 3D synthetic cell culture substrate, stem cell therapy, injectable therapies, biosensor development, high-throughput screening, biofunctionalized surfaces, printing biofabrication, and gene therapy.

21. A method of wound treatment comprising:
bioprinting a photocrosslink pattern to a subject using an apparatus comprising:
an inverted microscope sample stage;
a photomask;
a light diffuser;
a light pipe; and
a light source,
wherein the light source is confocal with an inverted microscope,
wherein the apparatus produces a ruthenium-mediated photocrosslink pattern into the subject, and
applying the photocrosslink patterned subject to an individual.

22. The method of claim 21,
wherein the photomask is disposed between the inverted microscope sample stage and the light diffuser,
wherein the light diffuser is disposed between the photomask and the light pipe,
wherein the light pipe is disposed between the light diffuser and the light source, and
wherein the light source is disposed above the light pipe.

23. The method of claim 21, wherein the individual is selected from the group consisting of a mammal, a reptile, a bird, a fish, an amphibian, and an invertebrate.

24. The method of claim 21, wherein the individual is a human.

25. The method of claim 21, wherein the subject comprises a hydrogel.

26. The method of claim 21, wherein the subject comprises a group consisting of a collagen hydrogel and a gelatin hydrogel.

27. A kit comprising:
an effective amount of a material, wherein the material comprises:
a photocrosslink patterned subject produced by an apparatus comprising:
an inverted microscope sample stage;
a photomask;
a light diffuser;
a light pipe; and
a light source,
wherein the light source is confocal with an inverted microscope, and
wherein the apparatus is configured to produce a ruthenium-mediated photocrosslink pattern into the subject.

28. The kit of claim 27,
wherein the photomask is disposed between the inverted microscope sample stage and the light diffuser,
wherein the light diffuser is disposed between the photomask and the light pipe,
wherein the light pipe is disposed between the light diffuser and the light source, and
wherein the light source is disposed above the light pipe.

29. The kit of claim 27, wherein the subject comprises a hydrogel.

30. The kit of claim 27, wherein the subject comprises a group consisting of a collagen hydrogel and a gelatin hydrogel.

31. A wound dressing or wound healing agent comprising:
a photocrosslink patterned subject produced by an apparatus comprising:
an inverted microscope sample stage;
a photomask;
a light diffuser;
a light pipe; and
a light source,
wherein the light source is confocal with an inverted microscope, and
wherein the apparatus is configured to produce a ruthenium-mediated photocrosslink pattern into the subject.

32. The wound dressing or wound healing agent of claim 31,
wherein the photomask is disposed between the inverted microscope sample stage and the light diffuser,
wherein the light diffuser is disposed between the photomask and the light pipe,
wherein the light pipe is disposed between the light diffuser and the light source, and
wherein the light source is disposed above the light pipe.

33. The wound dressing or wound healing agent of claim 31, wherein the subject comprises a hydrogel.

34. The wound dressing or wound healing agent of claim 31, wherein the subject comprises a group consisting of a collagen hydrogel and a gelatin hydrogel.

35. A pharmaceutical composition comprising:
a photocrosslink patterned subject produced by an apparatus comprising:
an inverted microscope sample stage;
a photomask;
a light diffuser;
a light pipe; and
a light source,
wherein the light source is confocal with an inverted microscope, and
wherein the apparatus is configured to produce a ruthenium-mediated photocrosslink pattern into the subject.

36. The pharmaceutical composition of claim 35,
wherein the photomask is disposed between the inverted microscope sample stage and the light diffuser,
wherein the light diffuser is disposed between the photomask and the light pipe,
wherein the light pipe is disposed between the light diffuser and the light source, and
wherein the light source is disposed above the light pipe.

37. The pharmaceutical composition of claim 35, wherein the subject comprises a hydrogel.

38. The pharmaceutical composition of claim 35, wherein the subject comprises a group consisting of a collagen hydrogel and a gelatin hydrogel.

39. A device for delivering a photocrosslink patterned subject to an individual, wherein
the photocrosslink patterned subject is produced by an apparatus comprising:
an inverted microscope sample stage,
a photomask,
a light diffuser,
a light pipe, and
a light source,
wherein the light source is confocal with an inverted microscope, and
wherein the apparatus is configured to produce a ruthenium-mediated photocrosslink pattern into the subject.

40. The device of claim 39,
wherein the photomask is disposed between the inverted microscope sample stage and the light diffuser,
wherein the light diffuser is disposed between the photomask and the light pipe,
wherein the light pipe is disposed between the light diffuser and the light source, and
wherein the light source is disposed above the light pipe.

41. The device of claim 39, wherein the subject comprises a hydrogel.

42. The device of claim 39, wherein the subject comprises a group consisting of a collagen hydrogel and a gelatin hydrogel.

43. The device of claim 39, wherein the individual is selected from the group consisting of a mammal, a reptile, a bird, a fish, an amphibian, and an invertebrate.

44. The device of claim 39, wherein the individual is a human.
